(19) United States
(12) Patent Application Publication
(10) Pub. No.: US 2015/0358452 A1
(43) Pub. Date: Dec. 10, 2015

(54) Additive Equipment to Multimedial, Health and Sport Equipment

(71) Applicant: Vladimir KRANZ, Prague 4 (CZ)

(72) Inventor: Vladimir Kranz, Prague 4 (CZ)

(21) Appl. No.: 14/433,385

(22) PCT Filed: Oct. 7, 2013
(86) PCT No.: PCT/CZ2013/000124

§ 371 Date: Apr. 3, 2015

(30) Foreign Application Priority Data
Oct. 5, 2012 (CZ) PV 2012-680
Jan. 3, 2013 (CZ) PV 2013-9
Mar. 4, 2013 (CZ) PV 2013-161
May 7, 2013 (CZ) PV 2013-337

(51) Int. Cl. H04M 1/725; A61B 5/0245; A61B 5/0404; A61B 5/0408; A61B 5/00; A61B 5/044; A61B 5/04; A61B 5/0452

(52) U.S. Cl. CPC H04M 1/72527; A61B 5/04012; A61B 5/0245; A61B 5/0452; A61B 5/04085; A61B 5/6823; A61B 5/6831; A61B 5/0006; A61B 5/6824; A61B 5/044; A61B 5/04087; A61B 5/6898; A61B 5/0404

(57) ABSTRACT

An addition of auxiliary functions to the multimedia equipment not included inside. These functions can be function of physiological data processing, extended and/or uninterrupted operations with regard to monitored and processed data. Auxiliary functions are implemented by the auxiliary equipment and circuits solutions physically placed in the original equipment or out of it but mechanically and electrically connected by it, whereas can it formed one compact unit. The parts, which are necessary to be dining operation changed to get uninterrupted functions, are user friendly and simply exchangeable from the aspects of users

The invention concerns the functions extension of multimedia equipment by auxiliary devices situated on them or separately. Solutions are elaborated also for sensing data of heart rate and transferring it into portable ECG or into a small size device for imaging of data or curve of heart rate, possibly on mobile phone placed on detachably on bracelet with respect to deployment of electrodes and cables on the body of checked person so as they do not bother to ordinary motion and activity. It should be also easy for installation, bearable from esthetic point of view and bearable and suitable for non interrupted sensing for unlimited time,

If multimedia is a cellular phone, it should be so called smartphone with software and hardware outfit ensuring very broad range of abilities and applicability, but on the other baud their power consumption is much higher than former similar phones. As a big drawback of these smartphones is insufficient power capacity of the accumulator.

The drawback is removed by an auxiliary device to the multimedia or health or sport equipment, where the multimedia equipment is with advantage a cellular phone or a tablet or transferable or pocket phone, medical or sport equipment, or with advantage monitoring, surveillance or diagnostic facility or detectors of heart rate or diagnostic chest straps for health or sport purposes, where multimedia equipment enables to use further described functions, or some of it such as are voice or/and data communication using mobile phone or other wireless network, multimedia playback, taking snaps, or using video recorder, receive TV or/and radio broadcast, GPS determination, and contains basic units and/or parts such as control elements comprising with advantage by push buttons or/and keyboard, or/and touch display, microprocessor unit, basic accumulator and other basic units allowing functions mentioned above, where auxiliary device contain at least one block consisting of auxiliary mechanical, electric and electronic modules, units and/or parts and with advantage of control elements and mechanisms for manipulation with accumulator formed by placement mechanism of accumulator and with advantage with mechanism for removing accumulator, which is connected to electronic device mechanically, electrically and electronically. With advantage it can be connected to multimedia equipment, which extends basic functions of multimedia equipment with additional functions which an auxiliary device situated inside can with its additional modules, units or parts allows to use where multimedia, equipment works perfectly with the all original functions as it was designed and produced. In removable version and paired version of auxiliary device connected to multimedia equipment non adapted from the factory for connecting with the auxiliary device, is the device removable connected mechanically to the surface of the non adapted multimedia equipment instead of the back cover situated ordinary on the opposite side that the display or keyboard which serves to cover parts such as the basic accumulator and/or SIM card and/or other parts the exchange o them the cover is removed, where the connecting mechanism of auxiliary device with the electronic circuits is formed by lug mechanism of auxiliary device with lugs and with advantage with fixing mechanism of auxiliary device with fixing elements with advantage fixing elements of auxiliary device are paired with fixing elements for cover of multimedia equipment. In general-purpose version the auxiliary device is removable connected to the surface of multimedia equipment non adapted from the factory for connecting auxiliary device with fixing elements with advantage with a clip, Velero, screws or other elements.

Another possibility of connecting the universal version of auxiliary device is to fix to the cover of multimedia equipment non adapted for fixing of auxiliary device with which is removed and replaced with a spare cover of multimedia equipment, or with another installed device, with advantage with other functions than removable auxiliary device, where the fixing elements are with advantage formed by depressions, clips, joining layers, Velero, glue layer, or fixing by glue, screws, or other suitable manner or by combination of these manners. Advantage of fixing of auxiliary device to the cover of multimedia equipment is the availability of covers which are sold as a snare parts and fixing auxiliary device on them allows easy the possibility of connection of one or more types of auxiliary devices to multimedia equipment by simply exchange of basic cover. The advantage of universal version is induced by the possibility of connecting the universal version of auxiliary equipment to any serially produced multimedia device, which needs not to be adapted in the production so as to respond to this connection, preferably the electrical connection will take place as; well simultaneously with the mechanical connection of the auxiliary equipment to the multimedia device by plugging-in the connector of auxiliary equipment to the connector of multimedia device, or the connectors are connected additionally. In that case, the U connector connecting the multimedia device with the auxiliary equipment connected thereto will preferably be used. Two connectors, one for multimedia device, the other one for the auxiliary equipment interconnected by the cable will preferably be used to avoid that the placing of connector on the auxiliary equipment need to be adjusted to the location of connector of the multimedia device. The partial auxiliary equipments may have different additional functions and they may preferably be engaged according to the need required by various degrees of extension of basic functions of multimedia device. Preferably, the auxiliary equipments will be mounted on the multimedia devices manufactured without adjusting the connection for the auxiliary equipment, i.e. they include the fastening elements for the cover only, rather than the auxiliary equipment whose connection thereto is not foreseen by the production. Preferably: all electrical and electronic units and parts of additional functions are placed in the auxiliary equipment. Tins advantage may apply particularly, when the purchase of multimedia device did not require the additional functions as they remained available, contrary to the situation of having them possibly installed in the multimedia device as the units and parts for the basic functions only are purchased. The advantage is to place the auxiliary equipment to the serially produced multimedia device, where all the electronics is contained in the auxiliary equipment to enable the costs reducing purchase of the multimedia device, unless the auxiliary equipment that might be acquired any time later was purchased simultaneously. In comparison to the devices enabling to install the auxiliary equipment, however, the units thereto are partly or fully placed in the multimedia device so as the units will increase the cost of multimedia device, if the auxiliary equipment was not purchased. There is also an advantage, in comparison with the multimedia devices, having the auxiliary equipment designed as a permanent installation, with the absence thereof the multimedia device either fails to be operational fully, or in case it was not engaged, the multimedia device would give an optical impression as it was incomplete, or non-compact. In case the auxiliary equipment was not used, its value may unnecessarily increase the cost of multimedia device as the multimedia device is not comprehensive without the latter. The fastening elements, preferably shaped in the form, of lugs to enable fixing the cover on the multimedia device, are folly or partly used for the removable holder of the auxiliary equipment equipped with the pieces vice versa placed to the lugs on the media device. They may preferably be amended or replaced by other lags mounted, additionally on the multimedia device to enhance mechanical connection of the auxiliary equipment. The use of serially produced original holders of multimedia devices is an advantage, or they are subject to adjustment, or amendment, or they are additionally replaced upon the production, which enables the costs reducing serial production of the multimedia devices regardless the intended use for the connection to the auxiliary device that allows for the mass production, and thus less cost consuming in comparison to the multimedia devices, whose connection to the auxiliary equipment has to be taken into consideration even in the production by other way than replacing it for the cover, or the fastening elements additionally mounted thereto, and it has to be equipped with the appropriate fastening elements even in the production, thus increasing the cost, and so it is not produced for wider use, and it is therefore suitable only for applications with the auxiliary equipment. In other version of detachable auxiliary equipment, it is installed to the adapted multimedia device for the specific auxiliary equipment produced to this end, and together with the latter it tonus a common device, as it is foreseen even in the production that the auxiliary equipment will be used, together with this multimedia device, or at least most of the time, and be subject to selling it simultaneously with the latter. By this, although the advantage of savings will be lost in comparison to the auxiliary equipment fixed to the serially producing multimedia device, without modification of the auxiliary equipment possibly defined as an optional auxiliary equipment, however, on the other hand, a number of advantages will be retained, particularly the capacity enhancement of the basic accumulator, and a possibility of its replacement without removing the auxiliary equipment, and further enhancing the functions of electronic device, preferably the multimedia device. The principles or co-operation and the advantages of specific auxiliary equipment and the multimedia device and the engagement of blocks remain basically identical to the paired or the universal auxiliary equipment even in the production. In case the auxiliary equipment is not connected thereto, preferably the exposed parts of electronic device, preferably by the multimedia device, may preferably be covered by the specially manufactured cover. As the simultaneous engagement of auxiliary equipment and the multimedia device is foreseen, the part thereof or all the electronics needed for the functioning of auxiliary equipment may be placed in the multimedia device. This is an advantage, when more pieces of auxiliary equipment with different additional functions apply to expand the multimedia device as required. By doing this, the savings wall be made in purchase of the electronic units and the parts thereof that are commonly utilized by more pieces of the auxiliary equipment, compared to the situation, when they were placed in all pieces of auxiliary equipment. The use in the auxiliary equipment is preferably possible for the removable auxiliary equipment of other control elements, whose conditions are detected by an additional microprocessor unit and transmitted to the basic microprocessor unit. The emergency call push button, a mechanical one large enough and visibly available in the auxiliary equipment may preferably be used in this way so that the person in danger may immediately make an emergency call, without complicated manipulation on the display, or keyboard of the multimedia device, preferably composed of the mobile phone, where the touch keyboard, in particular sometime requires the complicated manipulation to display the appropriate functional push button in the situation, when the push buttons are small and placed densely next to each other. Similarly, the reset push button may also be placed in the auxiliary equipment. In an integrating design, the auxiliary equipment is built in the compact multimedia device without a possibility of disconnecting it easily. Though, an advantage of price reduction is lost in case all, or some added modules, units, or parts thereof were not needed in purchase of the multimedia device, as they are now built in the compact multimedia device of a specific version, and upon the production they cannot be easily changed by engaging the auxiliary equipment equipped differently, however, the advantage of the advanced features in the compact multimedia device enabled by the additional modules, units and/or the parts thereof, contrary to the functions enabled by the basic units of the common multimedia devices, remains. In particular, it includes the power source capacity enhancement, actually an operating power source consisting of a basic accumulator, by the extra accumulator, easily replaceable in the operation of multimedia device without removing the cover of electronic device, preferably equipped with the multimedia device that is enabled by the accumulator handling mechanism, preferably the placement mechanism installed in the multimedia device firmly. Furthermore, it includes the engagement of two or more additional microprocessor units, communication block and the front end unit to process the BCG curve and other auxiliary units and parts thereof as described under the detachable auxiliary equipment. Other version includes the auxiliary equipment detachable from the compact multimedia device, where the auxiliary equipment equipped differently can be replaced. The disassembling is more complicated than in case of the detachable auxiliary equipment, but the advantage of using auxiliary equipment equipped differently, as needed, is induced, and by this also the advantage of cost reducing purchase price, as only the auxiliary equipment equipped appropriately is purchased, or no auxiliary equipment has to be installed. The detachable auxiliary equipment, preferably located in the PCS is detachable separately, or together with the PBM, units and the parts of compact multimedia device. The removable auxiliary equipment preferably takes the form of a cassette retractable into the body of a compact multimedia device. An easy replacement of the removable auxiliary equipment for the other one, equipped differently, is an advantage. The entire, or part of the auxiliary equipment is removable. The accumulator manipulating mechanism preferably includes the accumulator placement mechanism, or preferably the accumulator removing mechanism. For all types of the compact multimedia device, the auxiliary accumulator placement mechanism is preferably used to enable an easy replacement of the discharged accumulator for the recharged one during the operation of the compact multimedia device to charge the basic accumulator without removing the cover, in case of the serially produced multimedia devices, the auxiliary equipment allows adding the additional features, preferably to increase the capacity of basic accumulator by the capacity of additional accumulator in the auxiliary equipment, in this function, the auxiliary accumulator, through the appropriate electronic unit, preferably composed of a voltage converter, or a regulator, charges the basic accumulator of multimedia device via the electrical interconnections of the auxiliary equipment with multimedia device. The auxiliary equipment preferably enables to replace easily the auxiliary accumulator without removing foe whole auxiliary equipment by use of the accumulator placement mechanism, preferably composed of small door, or by mean of small lever, which simultaneously fixes the accumulator at the moment of inserting it, or by mean of the squeeze responding mechanism, when the inserted auxiliary accumulator is pressed by the squeeze, and by doing this, it is released and ejected, and fixed by the latch at the moment of re-inserting it. This advantage is also evident, contrary to the standard multimedia device without auxiliary equipment, where the accumulator replacement turns to be difficult and time consuming due to the need of removing the cover, which keeps it longer in terms of time. The basic accumulator preferably supplies the power to the multimedia device during the replacement of auxiliary accumulator so that this replacement occurs without interrupting the operation of mobile device. The advantage of easy replacement of the auxiliary accumulator is further supported by the ability to recharge several auxiliary accumulators in the same timers, which can be carried in the pocket or pouch upon recharging it, and available for replacement of the discharged auxiliary accumulator in the auxiliary device connected to the multimedia device during the operation, which induces the advantage of any extension of uninterrupted operation. For discharged accumulator it means that in the field it is not required to seek for an uneasy possibility to replace it by removing the rear cover of multimedia device and interrupting its operation, or replacing the entire auxiliary equipment. This means savings due to the tact that in case of replacing the entire auxiliary equipment, for sake of replacing the discharged accumulator, the other auxiliary equipment must be kept in reserve for this purpose, but the auxiliary equipment is more expense c and larger than the accumulator also due to the fact that it also contains the electronics for charging, or other additional functions. The costs reducing solution is to keep in reserve and exchange only the auxiliary accumulators provided by the solution of the present invention. Placing the charging electronics in the multimedia device, as applicable in the current state of technology, increases the costs of multimedia device, which becomes evident, when the auxiliary equipment is not purchased for the multimedia device. The solution specified in this invention, where all the electronics ensuring the functions of auxiliary equipment are located in the auxiliary equipment, thus representing the savings. Moreover, the solution of replacing the accumulator from the placement mechanism, preferably by small door, induces other advantage that the serially produced auxiliary accumulators may be used so as they can be utilized in their unchanged form of the specified utilization, thereby reducing significantly the purchase price of the accumulator compared to the price of accumulator, which would have to be manufactured specifically for this purpose, and so in much smaller series. In the auxiliary equipment, preferably outside the accumulator, other units and the parts thereof extending the original functions of the massively produced multimedia device can be placed. The utilization of one or more auxiliary microprocessor units, which preferably operates with different operating systems than the basic microprocessor unit of multimedia device, thus allowing not only to extend the performance of basic microprocessor unit, but also working with a wider range of operating systems, programs and applications, induces a particular advantage. This allows, for example, in relation to multimedia device comprised of the mobile phone with the Symbian operation system in the basic microprocessor unit to use the Android operation system in the additional microprocessor unit of the additional device, hence to enable the operation or programs and applications in both OSs. In the same time, by mean of communication via connector interconnecting the multimedia device and the auxiliary equipment, preferably through the USB port, or multi-pin connector, or by mean of interconnecting conductors in the auxiliary equipment integrated in the multimedia device, it is preferably possible to manage from the multimedia device the auxiliary microprocessor unit by use of the control elements in the multimedia device, and it also possible to display at the multimedia device the required data from the auxiliary microprocessor unit. It is preferably possible also to use the multiple microprocessor units in the auxiliary device, and thus increasing the number of operating systems. In comparison to the situation, when the auxiliary equipment was not used, the communication block is preferably placed in the auxiliary equipment to enhance the communication functions of multimedia device. The 5 kHz transmission module, and/or the bluetooth transmission module, ANT and/or the RF transmission module and/or the WiFi transmission module and/or the ZigBee transmission module and/or BT/BLC belt module and/or GSM communication module is preferably used as a communication block. The use of 5.5 kHz receiver for receiving the heartbeat pulses is an advantage so that it is possible to receive the heartbeat or other signals from the chest belt operating at the acoustic frequency, e.g. 5.5 kHz, which induces an advantage of lower power consumption by both the transmitter and the receiver. For example, the transmitter operating time of 5.5 kHz with a small watch accumulator of the chest belt operating at 5.5 kHz lasts tor about one year, which also applies for the receiver, compared with few hours of operating time of the chest belt operating in the bluetooth system. The reduced consumption also affects the power supply of the receiver, or 5.5 kHz transmitter in the auxiliary equipment. Other advantage of applying the 5.5 kHz signal for transmitting heartbeat is that this signal can be received by the multiple devices simultaneously, allowing the heart rate displaying simultaneously and the graphs and values at the multiple devices derived from its waveform and the values. To interconnect the chest belt with the electronics board is possible galvanicly by mean of the connecting element, preferably by the cable, or wireless, by mean of bluetooth. The output from the electronics board is connected to the display of auxiliary equipment via the connecting coupler. The microprocessor unit is also connected with the memory card and also with the module intended for the communication with a mobile data and/or voice network. By doing this, the multiple use of data taken is achieved for immediate checking in the watch by the monitored person on one side, or in the display of the auxiliary equipment of multimedia mobile device on the other. In the same time, the data taken and noted are permanently recorded In the memory card, and thus subsequently retrievable and assessable.

A possibility of utilizing the module for communication with a mobile data and/or voice network provides for an opportunity of transmitting data received from the distance server or the surveillance center respectively, with a possibility of distant monitoring, but also potential voice communication with the surveillance organ. The equipment designed in this way includes an unique multiple application as follows:
Heat Activity Recording Holier without access of the monitored person;
Monitoring and recording equipment for the monitored person with a visible output of curves and values in the display of watches, or multimedia device and simultaneous handing over this data to the surveillance centre;
Monitoring and recording equipment for the surveillance center with a potential wireless data transmission, but also the voice signal between the center and the monitored person.

This is particularly preferable in the situation of a simultaneous utilization of the wrist watch to display the heartbeat and other functions derived therefrom together with displaying the identical, or the adjusted data in the multimedia device to be larger, as a rule, than the wrist watch so that the heartbeat and the data deriving therefrom may operatively be continuously monitored in the display of the watch wrist band and in the same time in the multimedia device, preferably composed of a mobile phone receiving this signal in the same tune, may be put in the pocket, or a small bag, and it may be pulled out and switched on to enable the actual monitoring in case of need of a detail information only, if the display in the wrist band shows the values applicable to pursue the monitoring, particularly in case they exceed the limit and fail to provide comprehensive data compared to those provided by multimedia device equipped by larger display nut in the pocket. Similarly, the enlargement by other communication units working preferably in the ANT, the WIFI system and other BT units and units of other communication systems and the media may be used. Preferably, the auxiliary microprocessor and basic microprocessor are co-operating via the data interconnection by mean of the connector connecting multimedia device with the auxiliary equipment, preferably with the USB, or the multi-pin connectors, or interconnected by the conductive connections in the auxiliary equipment in the multimedia device to enable the use of other functions in the auxiliary equipment. Preferably, one of the functions of microprocessor unit of the auxiliary equipment will include foe EGG with the signals taken by electrodes pre-processed, in the front end part situated preferably in the chest belt, and transmitted via the Bluetooth or a similar communication signal to the communication block in the auxiliary equipment with tins signal being processed by the auxiliary microprocessor unit and transferred through the data connection to the basic microprocessor unit for further processing to display the ECG curve in the display of multimedia device, where the display control and other functions can be performed from the display, or the keyboard of multimedia device. By this, the ECG curve monitoring and their values and other evaluation are preferably enabled. The ECG mobile capability continuously displayed, and received and processed in this way, is an advantage, and the monitored person may carry the device in her/his pocket. According to the need, she/he may check her/his conditions, particularly in the tune of despatching the emergency signal in case the pulse, or the ECG, or other monitored health functions preferably detected and transferred by the chest belt, are beyond the limit. The ECG processed by the auxiliary equipment represents a similar solution, where the electrodes are connected by the cable with the auxiliary equipment inducing the advantage of a higher reliability and the elimination of an interference compared to the transfer via the BT, while the ECG may be processed in the auxiliary equipment and controlled and displayed in the multimedia device. In this case, the front end part is placed in the auxiliary equipment. The ECG and other medical data, or other data are preferably transferred from the auxiliary, or multimedia equipment via the wireless data network even to the distant server, preferably located in the surveillance centre, or other data storage, wherefrom they can be displayed any time, preferably in the PC locally, or on remote over the data network. The auxiliary equipment can preferably be placed in the multimedia device composed of a mobile phone, or in the medical equipment placed in the wrist belt so as all features described preferably apply in this case as well. The connection with the basic equipment is preferably pursued via the USB connector on the wrist band, or other parts of a mobile phone. The auxiliary equipment is mounted on the wrist hand in the direction from, or towards the body, or under the mobile phone. In case of the version led from part of the body, it is preferably led from the connector placed in the wrist band led by the USB to the case of the auxiliary equipment. In case of the version close to the body, it is preferably fed from the connector placed, as in case of the version led from part of the body, by mean of the interconnecting element, preferably by the cable to the case of auxiliary equipment, whereon it is placed, in case of the version under the mobile phone, it is also preferably led by the cable from the connector as described earlier. In case of the tailor-made multimedia device, the cable is built into the multimedia device, or to the wrist band so that it is not visible. The interconnecting element is preferably composed of the cable, and/or twisted cord, and/or coiled cable, and/or wires. The case of auxiliary equipment preferably includes the placement mechanism for the auxiliary accumulator that can be pulled out easily through the small door without interrupting the operation. The additional accumulator charges preferably the basic accumulator. The printed circuit board with the blocks and units of electronic equipment, preferably the microprocessor units and communication blocks and other units placed thereon, ate located in the said case, and they are preferably removable so that the blocks equipped, as required, can be used.

If the USB connector was located in the other part than the wrist band, the basic equipment is then connected to the auxiliary equipment appropriately by mean of the cable. On the surface of the case intended for the auxiliary equipment in case of the version close to the body or under the hand, which is placed in the bottom part of the wrist band, there is a display preferably placed that may display the data processed by one or more microprocessor units m the auxiliary equipment, or from the basic equipment. Preferably, one or more microprocessor units and other units are controlled by controlling elements of a mobile phone, or they have their own control elements. This is particularly preferable for the panic, or reset buttons, which are preferably located on the case of auxiliary equipment, thus enabling them to be of a larger size and clearly separated from other control elements, and thus controlled in case of emergency more easily and without error, call for help and/or reset in case of pressing the panic button by mistake, and/or in case of emergency signal due to health functions outside the limit, if evaluated so that the alarm to the surveillance center should not be transmitted. The multimedia equipment in the wrist band is preferably located on the sliding mechanics, which is preferably adapted to remove the multimedia device easily that allows to slide out the media device towards the band, when the display needs to be watched, and/or push it back towards the forearm so that it can be hidden under the shirt in case the display was not necessary to watch. The multimedia device, preferably comprised of the wrist baud mobile phone, may apply to evaluate the ECG, which is preferably placed on the chest belt. The connection is preferably made via the bluetooth transmitted from the chest belt, where the ECG unit, including the front end part, is placed, or it is interconnected by mean of the multimedia equipment on the wrist band via the cable connected to the connector. In that case, the trout end part may be placed in the auxiliary equipment next to the microprocessor unit located in the wrist band mobile phone and the detectors placed on the body may be connected by the connecting element, preferably by the cable. In case of a wire connection, the comprehensive ECG control unit may be placed in the wrist band mobile phone and the electrodes remain on the body, preferably and partly placed in the chest belt. The ECG may be placed in the special unit preferably placed in the wrist band next to the wrist band mobile phone and interconnected, via the cable, or the bluetooth, to the chest belt and electrodes. It is interconnected to the wrist band mobile phone via the cable of connectors, or the Bluetooth, or other suitable communication media by mean of the communication blocks. Taking the heartbeat pulses is preferably performed by the five electrodes system, where three electrodes are preferably located in the chest belt under the breast nipples and two additional electrodes in the area of shoulder holes, preferably placed in the braces, are connected with the belt by the cable, preferably due to the aesthetic aspect covered by the braces. The three leads ECG may be achieved by this version. The single lead BCG recording, two electrodes located in the chest held under the breast nipples may apply, where the third electrode ensuring the zero nominal voltage is preferably added in the middle. To enhance the display of P wave, one more electrode is preferably placed in the shoulder hole and connected by the cable to the chest band, preferably by the connector so that it can be disconnected, if the P wave should not be enhanced. In case of both, the one and three leads version, the cable to the wrist band, or the adjacent ECG unit, leads from the band with all electrodes, preferably conducted to via the cable drawn around the neck and further on the arm. Upon getting the shirt, or blouse dressed, this provides for that neither the chest belt, nor the electrodes of distribution by the cables are noticed from outside, for taking the signals in corresponding quality, the electrodes with contacts fitted to by a layer of conductive gel are needed. For the strip mentioned above, the frame mounting electrodes system with those electrodes being newly designed and placed in the transportation cartridges, where the electrodes first are snap-fastened to the belt. Hereafter, as the belt is fixed around the body in the correct position, the transportation cartridge is removed with disclosing the contact surface fitted to by the gel layer. Subsequently, the belt presses the electrodes into the working positions on the surface of a body of the monitored person. The electrodes bonded to the conductive surface of the chest belt only are the option, when the adhesive ring ensures the gel leaks through the center hole at the magnified pressure to create a contact with the body of monitored person. The accumulator handling mechanism, preferably the accumulator placement mechanism allows for an easy accumulator replacement, preferably by the charged one, without opening or removing the accumulator cover, or the cover of electronic equipment, preferably the multimedia, or medical device, or the replacement of the entire auxiliary equipment, or the part thereof, preferably during its continuous operation using the charging and power supply electronics module, preferably consisting of the accumulator block, preferably added by other auxiliary electronics. The accumulator handling mechanism preferably in the electronic device, the block of power supply of the electronic equipment may be subject to replacement during the replacement of the basic accumulator so as they can be used in various, preferably newer models of multimedia, or medical, or auxiliary equipment to save the purchase cost by mean of use those stemming from the older model. The replacement may preferably be performed by inserting, screwing, snapping avid inserting it simultaneously in the male/female connector. The accumulator handling mechanism is preferably based on the accumulator small door snap fixing principle, or the accumulator snap automatic releasing principle, or the accumulator automatic sliding in lock and the accumulator repeating push release, where the outer side of accumulator, in the same tune, creates a wall to cover the hole for inserting the accumulator, if the door was not engaged. The door hinges are preferably made of metal or plastic, and they are preferably equipped by rotatable hinges, or the hinge is preferably formed by a bendy resistant material, preferably plastic, used for the remaining part, and thinned appropriately in the area of hinge. The contact to the conductive part of the flexible elastic, shapeable material, preferably serves the EGG electrode, or the heart rate monitor, preferably with the conductive gel, i.e. the gel electrode. The electrode is mounted on the clamping element, a chest or a foot one, or the braces, or by mean of the plastic clamping garment, or the harness, preferably removable and replaceable with a new electrode by the electrode fastening mechanism with the latter being preferable created on the fastener principle, or by the adhesive foil bonding, or in other suitable way. The advantage of placing the electrode with the electrode fastening mechanism at the clamping element is induced by the fact that the electrode is not bonded to the skin, as noted by the current state of the technology indicating the disadvantage of a potential allergic reactions and painful withdrawal, albeit attached to the pressure belt, preferably the elastic one, pressing it to the skin by pressure preset suitably to achieve a reliable contact increased by the conductivity, or the adhesion of a preferably gel material able to be removed from the skin easier than the glued contact. The placement mechanism for additional parts makes it possible to use the identical additional part in the multiple models, preferable newer ones, thus saving the purchase costs. The removable lugs are made preferably by sliding in, or snapping to, or screwing by, or gluing to, or in other suitable way. The auxiliary equipment is mounted on the multimedia equipment by mean of the auxiliary equipment connecting mechanism, preferably by the auxiliary equipment lugging mechanism. This enables to lung the auxiliary equipment to the various models of multimedia equipment, preferably also to the newer models to induce the advantage of making it possible to use the current equipment, if the other model was selected, thus saving the purchase costs particularly by changing it for a new model. Mounting the auxiliary equipment by screwing, or gluing to the cover of multimedia equipment serves another example of connecting the auxiliary equipment, where the irregularities of surface of the cover are subject to the application of a flexible underlay, if mounted by screws, or glued. The size of auxiliary equipment is preferably smaller than the multimedia equipment, thus it may be worth for use of a large spectrum of models of the multimedia equipment. The electrical interconnection is preferably made by mean of the interconnecting element, preferably the cable on one side equipped by the connector, preferably containing the USB, that uses to be inserted into the connector of multimedia equipment with the other end led from the multimedia equipment containing the hidden reserve of the cable to enable the adjustment for various models of multimedia equipment. In the current state of technology, the chest belts intended to take the heartbeat pulse, or the ECG, induce a disadvantage of jam incurred due to the body movement, when the part of belt surrounding the chest moves that part of the belt which contains the electrodes, thus sliding on the underlay causing the jam. This malfunction is subject to removal by the sliding chest belt, preferably composed of the scanning part with the electrodes and the fixing part surrounding the chest and sliding freely on the fixing part, whereto it is not connected mechanically, thus not moving against the skin during the body movements. For sake of better sliding on the surface, the sliding material, preferably a Teflon is used on both sides of the belt. The sliding belt principle is preferably also used in the braces connected to the chest belt with their elasticity pressing to the scanning part containing the electrodes placed in the area of aims to create, together with the electrodes on the chest belt, the system of electrodes tor the three leads ECG and by other two electrodes on the chest belt and two electrodes on the foot belt, 10 electrodes in total, can create the 12-leads ECG. The electrodes are connected by the interconnecting element, preferably the multi-strapped cable, to the electronics board service for processing the heart signals for the ECG, and/or the heartbeat pulse curve preferably placed in the small bag at the umbilical part of the body, or in the pocket, or on the wrist preferably drawn from the sleeve and preferably placed on the wrist band with the sliding mechanism enabling the possibility that it may be pulled out to achieve an easy handling. The interconnection to the board electronically is preferably made by the interconnecting element particularly the enhanceable cord composed of the twisted cord, or the coiler. The interconnection to other equipment, preferably the multimedia equipment, PCs and servers, is made wireless. The famous irregularities in discontinuing power supply during the replacement of basic accumulator is to the substantial extent removed by the accumulator handling mechanism ensuring that the basic accumulator is charged from the auxiliary equipment, preferably placed in the auxiliary equipment. An easy replacement of the accumulator for the recharged one, without interrupting the operation and without the need to remove the cover, is achieved. The placement mechanism is preferably composed of the small door with a small lever fixing in the same time, the accumulator during sliding it into the mechanism. The mechanism reacts to the squeeze, when the inserted additional accumulator is pressed by the squeeze, and thus released and slided out. In the repeating insertion, it is fixed by the latch. The accumulator mechanism particularly the placement mechanism, or the accumulator pulling out mechanism is equipped by the tilting, or the sliding small doors, or the tilting, or the sliding cover, or the part thereof or the entire auxiliary equipment may be tilted, or slided during the placement, or replacement of the basic, or the additional accumulator. Upon recharging the accumulators, an easy replacement of the additional accumulator provides for a possibility to recharge several accumulators in the same time, which can be worn as a reserve in the pocket, or hand luggage, and so may be available for the fast replacement of the additional discharged accumulator in the auxiliary equipment connected to the multimedia equipment without interrupting the operation of this multimedia equipment. The own accumulator of multimedia equipment is continuously recharged in this way in any operation place, anywhere in the field, in the transportation means, etc. The recharging of the own accumulator of multimedia equipment in this way is not necessarily subject to connection to the network charger, or the notebook, in addition to charging the own accumulator of multifunctional multimedia, equipment, the additional accumulator may also ensure power supply to this single equipment itself. In this way, the continuity of operation of electronics equipment, i.e. the multimedia equipment, preferably a mobile phone, is ensured without any and all doubts, and a possibility of operation failure due to lack of the operation power supply is excluded fully. The way of replacement of additional accumulator from the placement space of auxiliary equipment, preferably by mean of small door creates a special advantage particularly in enabling the use of the serially manufactured accumulators for the function of additional accumulators possibly applicable in the used and specified system composition, which may be utilized In the unchanged and non-adjusted way. In this way, an outcome is reached that in comparison with a possible use of accumulators intentionally adjusted for this purpose in the system designed differently, the purchase price of additional accumulators will be in no way increased. Such a specially adjusted accumulator would be produced in lower series, thus reasoning the higher costs than the accumulator of a standard version used in the described system. The accumulator handling mechanism particularly the placement mechanism of the additional accumulator, preferably the universal one intended for recharging the bask accumulator, preferably placed in the auxiliary equipment and an easy replacement without interrupting the operation of multimedia equipment and without taking away the rear cover. The placement mechanism is preferably created by a small door, which simultaneously fixes the accumulator at the moment of inserting it into the placement mechanism, which responds to the squeeze, when the inserted additional accumulator is pressed by the squeeze, and by doing this, it is released and ejected, and fixed by the latch at the moment of re-inserting it. Upon recharging the accumulators, an easy replacement of additional accumulator provides for a possibility to prepare and recharge simultaneously several accumulators by the external charger so as they may be worn in the pocket, or in the hand luggage to become gradually available to enable the fast replacement of the discharged additional accumulator in the auxiliary equipment connected to the multimedia equipment without interrupting the operation of this multimedia equipment. In this way, the own accumulator of multimedia equipment is continuously recharged anywhere in the field, transportation means, or free environment, etc., with the recharging accumulator being independent from connecting it to the network charger, notebook, or other power source, in this way, the operation continuity of the multimedia, or the monitoring equipment, without recharging them from the external source connected to the power distribution network, is ensured. In other design of the placement mechanism of accumulator, the tilting case with the additional accumulator is lifted so that the spring is partly slided forward to allow for taking it out easily. The closure of the case, not only unlocks the case, but also releases the additional accumulator lifting it from the contacts of multimedia, or monitoring equipment by other spring, and slides it out partly from inside the case. Other design of the placement mechanism of accumulator is preferably adjusted for use on the mobile phone west belt, or other multimedia, or monitoring equipment. In this case, the additional accumulator is fixed by the clamp and slided out partly by the spring upon release, in the other design, the frames beveled will preferably press the inserted additional accumulator into the case. In other version of the accumulator placement mechanism, it is fixed by the cradle-shaped latches opening in both directions, to release them preferably by the fingers, with the possibility of latches opening due to the bevel during the insertion of additional accumulator. The cradle-shaped latches are preferably extended beyond the joints and diverted in the angle from the case, thus allowing the latches opening by pressing the fingers to release the accumulator.

### BRIEF DESCRIPTION OF THE DRAWINGS

FIG. 1 Example of unmodified multimedia equipment formed by unmodified cellular phone

FIG. 2 Unmodified multimedia equipment formed by unmodified cellular phone with removed cover and with modified auxiliary device

FIG. 3 Detail of connecting modified auxiliary device

FIG. 4 Option of auxiliary device with small door situated on side

FIG. 5 Block diagram of a module for continuous charging of basic accumulator forming a part of auxiliary device

FIG. 6 Block diagram of auxiliary device, the electronics and all functions of which are placed on a hoard of electronics

FIG. 7 Block diagram of auxiliary device where on the board of electronics a current microprocessor is replaced by microprocessor with two USB ports

FIG. 8 Block diagram of auxiliary device extended for ECG signal processing

FIG. 9 Arrangements of auxiliary device for evaluation of ECG signals which are pre-processed by electronics placed close to electrodes

FIG. 10 Mechanical arrangement of auxiliary device and placement of its main parts

FIG. 11 Modified multimedia equipment with electronics designed commonly with specific auxiliary device

FIG. 12 Block diagram of continuous charging of basic accumulator

FIG. 13 Auxiliary device integrated into compact multimedia equipment

FIG. 14 Example of arrangement of main parts of compact multimedia equipment alternatively supplied from auxiliary accumulator

FIG. 15 Block diagram of a module of electronic for charging, placed on the board of electronic of multimedia equipment

FIG. 16 Example of modules placed on the board of electronic of compact multimedia equipment

FIG. 17 Example of simple storage mechanism of auxiliary accumulator

FIG. 18 Depiction of partly slicked out of accumulator from the case with opened small door.

FIG. 19 Another variant of storing mechanism

FIG. 20 Mechanical connection of unmodified multimedia equipment with modified auxiliary device with fixing elements formed by flexible clips

FIG. 21 Mechanical connection of unmodified multimedia equipment with unmodified auxiliary device with fixing elements formed by joining layer

FIG. 22 Mechanical connection of multimedia equipment with auxiliary device with fixing elements formed by mechanical joining elements with pins and pinned cavities with transverse springs

FIG. 23 Mechanical connection of multimedia equipment made by modified cellular phone with auxiliary device modified by inserting of grooves of multimedia equipment into grooves of auxiliary device

FIG. 24 Connection by fixing elements using connecting pins inserted into pruned cavities with transverse springs

FIG. 25 Mechanical connection with fixing elements realized by flexible lugs

FIG. 26 mechanical connection by inserting the auxiliary device into space made for this purpose in multimedia equipment

FIG. 27 Configuration of compact multimedia equipment with extended functions

FIG. 28 Compact multimedia equipment with extended functions, where auxiliary device is removable placed in multimedia equipment on separable exchangeable board of electronic

FIG. 29 Configuration of compact multimedia equipment with extendable functions

FIG. 30 Storing mechanism of accumulator reacting with clasp

FIG. 31 Option of modified multimedia equipment

FIG. 32 Compact multimedia equipment comprising mechanism of placing a block into multimedia equipment

FIG. 33 More simple mechanism of placing the board provided with a panel into the block of multimedia equipment

FIG. 34 Connection of unmodified auxiliary device with a bumpy cover of unmodified multimedia equipment

FIG. 35 Bracelet cellular phone with extended possibilities of using specific auxiliary device

FIG. 36 Block diagram of internal arrangement of cellular phone with extender possibilities of using splaced in case for fixing watch

FIG. 38 Option of specific auxiliary device allowing to fix the case of the equipment to opposite side of the bracelet

FIG. 39 Storing mechanism of accumulator with the board of electronic for charging control of the basic accumulator of bracelet cellular phone placed In case of auxiliary device

FIG. 40 Auxiliary device provided by storing mechanism for putting and connecting the charging auxiliary accumulator

FIG. 41 Situation after unlocking of tipping small door, which made the cover of storage mechanism

FIG. 42 Detailed view of mechanics and storing accumulator

FIG. 43 Placing the storage mechanism with tipping case in auxiliary device

FIG. 44 another example of mechanism of auxiliary accumulator for bracelet electronic equipment

FIG. 45 Storage mechanism of auxiliary accumulator in chest strap

FIG. 46 Option with storage device of auxiliary accumulator in chest strap with another fixing of baste accumulator

FIG. 47 Storage device of accumulator with arresting mechanism,

### DETAILED DESCRIPTION OF THE INVENTION

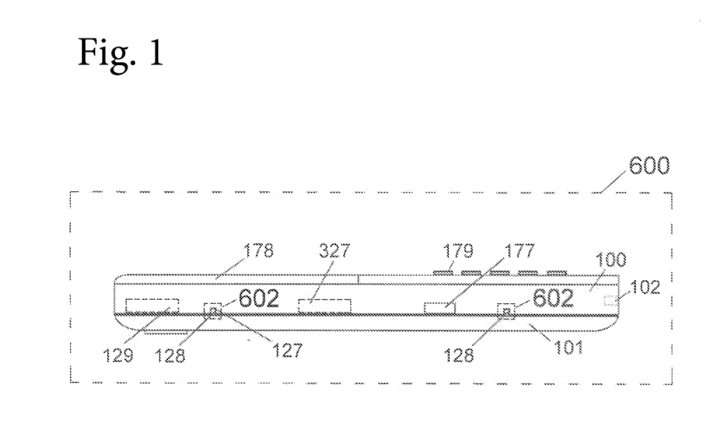

FIG. 1 presents example of individually unmodified multimedia equipment 600 formed by individually cellular phone 100 unmodified during the production for connecting of an auxiliary device. The removable cover 101 is joined with the cellular phone 100 by fixing elements 602 formed by lugs 128 of cover fitted into depressions 122 which are made by grooves on the cellular phone 100, where removable cover 101 is placed on reverse side of display 178 and keyboard 179 and coves SIM card 177 and basic accumulator 129 placed inside the cellular phone. On the cellular phone 100 is planed connector 102 for connecting USB cable, inside is placed basic accumulator 129, the basic microcomputer unit 327 of multimedia equipment, fee unmodified multimedia equipment 600 is made with advantage as well by multimedia pocket computer or similar multimedia equipment containing further described functions or some of them, such as mobile communication by means of internet, communication on mobile phone network or other data communication media, multimedia playback, photo-camera, TV camera, GPS detection with virtual keyboard equipped with keyboard, and display or touch display.

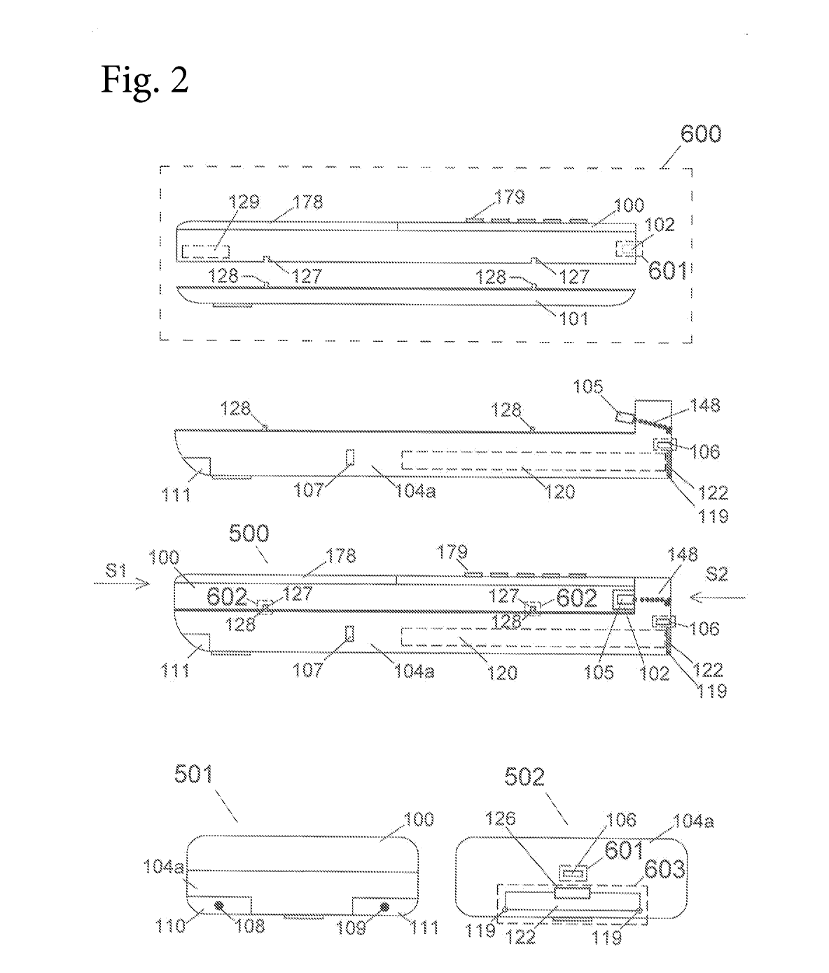

FIG. 2 shows unmodified multimedia equipment 600 formed by cellular phone 100 with disconnected removable cover 101 and modified auxiliary device 104a which extends functions of unmodified multimedia equipment 600. Connection of auxiliary device 104a to multimedia equipment 600 made of cellular phone 100 is constructed in the way, that auxiliary device 104a is placed instead removed cover 101. This solution with advantage allows its very easy mechanical, electrical and electronic connection/disconnection to/from multimedia equipment. In case when is not intention to use functions provided by auxiliary device, than is possible remove the auxiliary device 104a and instead of it to put the original cover 101 of multimedia equipment 600. With advantage it is also possible to use the auxiliary device in case, when is desirable to support the capacity of basic accumulator 129, or to use functions of the auxiliary device. There is not needed to purchase auxiliary device at the same time with the multimedia equipment, it can be purchased in case of later need. In such a case only the multimedia equipment is purchased, which leads to saving, because with advantage all electric, mechanic and electronic parts for operation, connecting and interface to multimedia equipment are situated in auxiliary device, so the acquisition value of the multimedia equipment is limned only on those units and parts multimedia equipment needs for us functions, which is lower than if multimedia equipment contained also part for cooperation. In this example, in the part of auxiliary device 104a which extends the size of cellular phone 100 there is placed second connector 105 USB, mounted with advantage using suspension element 148, which is during the mechanic connecting of auxiliary device inserted into connector 102 of cellular phone 100. When click, connector 102 is fixed with the second connector 105 which connects electronically the auxiliary device. The storing mechanism 603 of accumulator with the small door 122 for exchange of discharged 120 of auxiliary device 104a for charged one ensures easy approach to the accumulator and easy exchange and in the same time their locking by a lock 126 of door of accumulator fixes accumulator 120 against sliding out. On auxiliary device 104a is also placed another connector 106, with advantage USB connector, common for external communication and multimedia equipment, with advantage with external PC, which is used for charging voltage 5V for charging accumulator 120 of auxiliary device 104a from external source. With advantage can be used the change-over switch 107 data of external USB port for switching-over the data flow transferred by second connector 105 USB from processor unit, of auxiliary device, or alternatively from the next connector 106. Detail 500 shows a set of multimedia equipment formed by cellular phone 100 with connected auxiliary device 104a detail 501 shows a look at the set in S1 direction and detail 502 shows a look at set in S2 direction. Buttons 110 Reset and 111. Panic on detail 501 enable to pass on into multimedia equipment with advantage formed by cellular phone 100 with advantage commands, and indicators LED 108 with advantage red and LED 109 with advantage green, with advantage placed inside buttons 110 and 111, can signal reaction on these commands. With advantage, the button 110 for calling in emergency and button 111 for reset which is on easy accessible place at auxiliary device 104a have advantage for immediate use for calling in emergency in comparison with situation when these buttons are on touch display of cellular phone 100 and are accessible only after manipulation for wakening display. On detail 502 is a connecting element 601 made by another connector 106, further there are small door 122 with hinge 119 and a lock 126 of small door. It is easy to exchange the discharged auxiliary accumulators 120 with charged accumulators 129 in auxiliary equipment 104a without interrupting the operating status of the multimedia equipment 600 and to charge basic accumulators 129 thanks to storage mechanism 603 without removing of auxiliary device 104a from multimedia equipment 600 winch is with advantage formed by a cellular phone 100. In this way, there is not necessary to charge it with a relevant cable and connector 106. This connector remains still free. The user does not need to charge the cellular phone 100 through connector 102 from a charger and in this way be or she has the cellular phone 100 still ready for use. Another advantage of the auxiliary device is a lot of applicable functions supporting and extending the use of multimedia equipment 600. An advantage is the possibility to use unmodified multimedia equipment 600 from line production, which can be connected with auxiliary device 104a by exchange of original back cover 101 of the cellular phone 100 because multimedia equipment does not need any adjustment for connecting. When functions of auxiliary device are not used, the auxiliary device can be again replaced with the original removable cover 101 of the cellular phone. Connecting or disconnecting the auxiliary device 104a which can be done during operation of the multimedia equipment does not change the original functions of multimedia equipment. An advantage after connecting the auxiliary device is continuous uninterrupted operation of multimedia equipment 600 without the need of charging the basic accumulator 129 using an external charger, which is by auxiliary accumulators 120 placed in auxiliary device 104a, where can be used line production auxiliary accumulators available on market. Due to storage mechanism 603 is not necessary to use expensive battery pack.

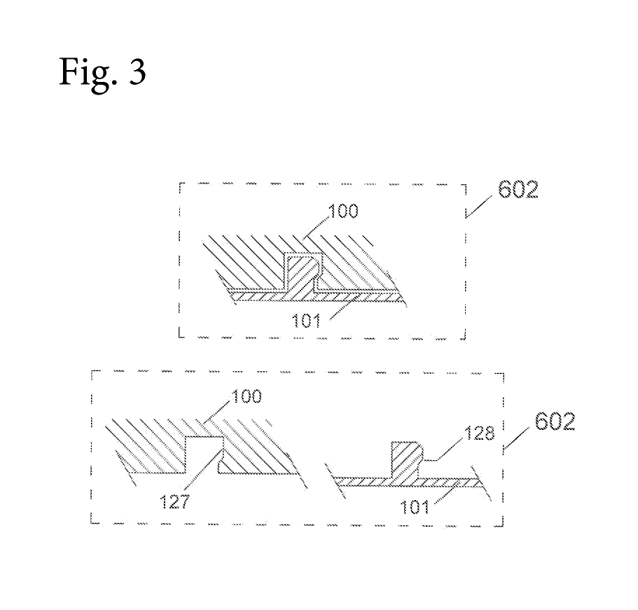

FIG. 3 is a partial cross-sectioned view illustrating mechanical connection of modified auxiliary equipment 104a by the use of fixing element 602, which is created by producing of grooves on the auxiliary equipment 104a the same as the grooves of unmodified multimedia equipment 600 created by the cellular phone 100, as presented, in the FIG. 2. The use of existing lugs on the multimedia equipment 600 unmodified is there illustrated. By the place of the auxiliary equipment 104a on the cellular phone 100 and mutual press down lug 128 of auxiliary equipment 104a click into place of counterpart 127 created by depressions in the cellular phone 100 and in this way is created strong but disassembling mechanical connection. By this reliable connection are both equipment reciprocally fixated, and so the position of them, units and parts inside are safe against accidental movement and ordinary handling by tins complex is possible. In this way created complex by the auxiliary equipment and multimedia equipment fixing is possible easy insert into case or pocket.

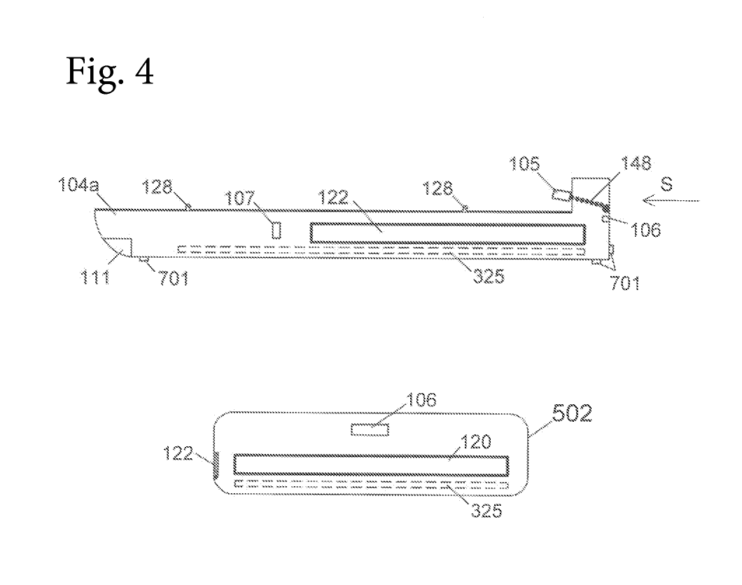

FIG. 4 illustrates variation of modified auxiliary equipment 104a with small door 122 placed on the side, which covers inserted auxiliary accumulator 120. Flexible fixing of second connector 105 to modified auxiliary equipment 104a by the use of flexible element 148 is without changes. The modified auxiliary equipment 104a display 325 is in addition illustrated, with advantage is touch display controlled by push buttons 704 secondary, which can be used to also for another purposes, where display 325 of auxiliary equipment makes possible continuous watching important information, for example the heart rate curve, simultaneously with other cellular phone 100 activity, see FIG. 1, where advantage is also possibility watch simultaneously two different activities on cellular phone 100 and auxiliary equipment 104a.

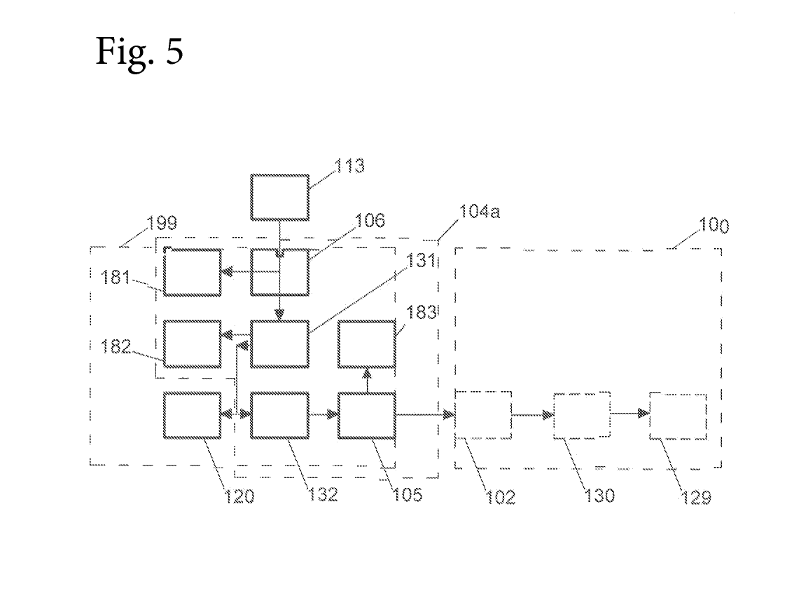

FIG. 5 illustrates block diagram of module 199 continuously charging of basic accumulator 129, which is part of modified auxiliary equipment 104a. This example illustrates charging of the basic accumulator 129 of cellular phone 100 and the auxiliary accumulator 120 at the same time from the charger 131, which simultaneously supplies the electric circuits of module 199 also multimedia equipment 600 formed with advantage by cellular phone 100. In this case external source 113 is not connected, it is going on at the same time recharging of the basic accumulator 129 of cellular phone 100 from the auxiliary accumulator 120, which is placed in the auxiliary equipment in the way, that is at first the voltage of auxiliary accumulator 120 modified by voltage converter 132 to voltage by the specification for charging multimedia equipment, frequently 5V. Via second connector 105 USB of auxiliary equipment 104a and to it connected connector 102 USB of multimedia equipment with advantage formed by cellular phone 100 is this voltage used for recharging of the basic accumulator 129 via charge 130 of multimedia equipment. The advantage of this solution is based on tact that it is possible to use in the auxiliary equipment accumulators with different voltages regardless voltage for recharging multimedia equipment accumulator. The auxiliary accumulator 120 placed in the auxiliary equipment inside is possible after discharging easily and quickly replace by another charged accumulator or recharge it from external source 113. Replacement of the auxiliary accumulator 120 of module 199, in which is placed auxiliary equipment inside is going on during operation without auxiliary equipment 104a cover removing and without operation interruption of multimedia equipment with advantage formed by cellular phone 100, since during auxiliary accumulator 120 replacement of auxiliary equipment, its operation is supplied by the basic accumulator 129. The auxiliary accumulator 120 can be replaced without operation limitation and without separation of multimedia equipment with advantage conned by cellular phone 100 and auxiliary equipment 104a. The auxiliary accumulator 120 placed in auxiliary equipment can be recharged from external source 113 which is connected via next connector 106 to the charger 131, which ensures recharging by the way of the auxiliary accumulator 120 specification. All electronics related to accumulator recharging is with advantage placed in auxiliary equipment 104a. The states of voltage during accumulator recharging which is placed inside auxiliary equipment and presence of voltage on the voltage converter output the indicators 181, 182 and 183 displays, of which circuits evaluate this voltages and with the advantage are monitoring this states and indicates by the use of LED of various colors. Indicator 181 signals presence connection of external voltage on next connector 106, in addition indicator 182 signals recharging of the auxiliary accumulator 120 in auxiliary equipment. Correct function of voltage converter 132 and thus indirectly state of auxiliary accumulator 120 which is placed in auxiliary equipment signals voltage indicator 183 for recharging basic accumulator 120 of cellular phone 100.

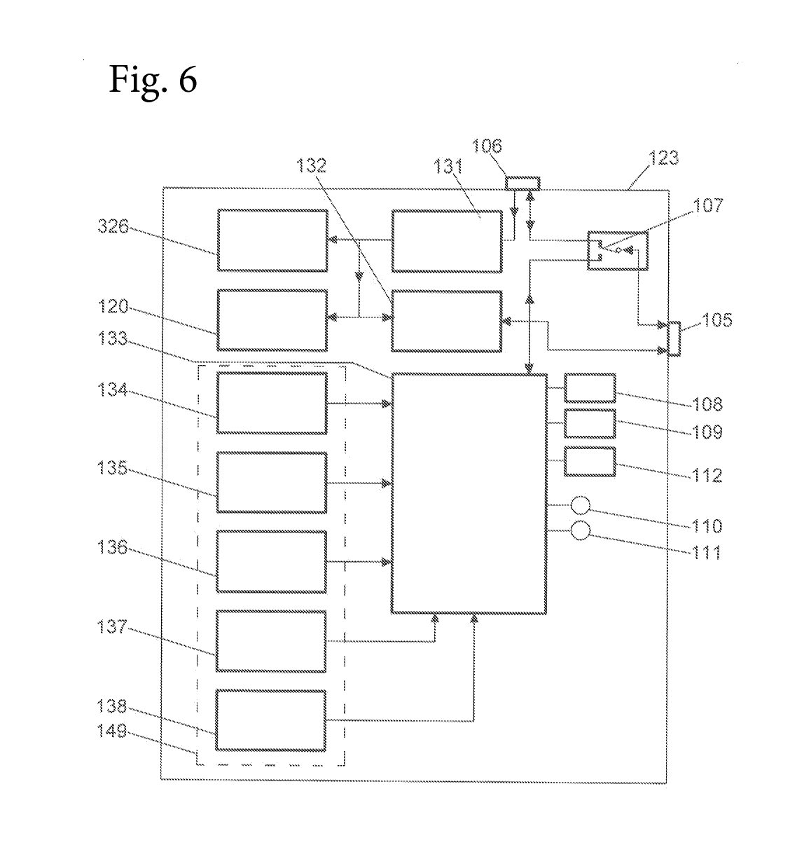

FIG. 6 illustrates block diagram of auxiliary equipment 104a, see FIG. 2 and FIG. 4, electronics and all functions of which are placed on the auxiliary equipment board 123 of electronics. Auxiliary accumulator 120 placed in auxiliary equipment 104a can be very easily replaced by the use of small door 122 for the auxiliary equipment without multimedia equipment operation interruption and without separation multimedia equipment and auxiliary equipment, is used for maxillary equipment supply via circuits 323, in which is auxiliary accumulator 120 placed and also used for charging of basic accumulator 129, which is placed inside the multimedia equipment. Auxiliary accumulator 120 can be in auxiliary equipment 104a placed on the board 123 of electronics or can be with this board connected and it is possible to recharge it without removing it from auxiliary equipment 104a by the catenae supply via next connector 106 external USB of auxiliary equipment and charger 131. Continuous recharging of the basic accumulator 129 of multimedia equipment 600 is from voltage converter 132 and link up to USB connectors 105 and 102. It makes possible to uninterrupted operation of multimedia equipment without necessity to recharge basic accumulator 129 by the external charger. The main function of multimedia equipment connector 102 USB is the possibility to connect cable USB for data transfer to the cellular phone, but which is covered due to mutual connection of multimedia equipment and auxiliary equipment and is being used by the auxiliary equipment apart from the tact that is used for recharging of multimedia equipment accumulator also for data transmission between the auxiliary equipment microprocessor 113 and multimedia equipment and thus are limited the possibility of multimedia equipment used the connector 102 for USB cable connection. The problem of multimedia equipment connector USB covering solve the data switch 107 of external USB, which switches data path multimedia equipment connector 102, which is inserted into the second connector 105, between microprocessor 133 and next connector 106 of auxiliary equipment, so next connector 106 can from the point of view of data transfer and USB cable connection fully substitute function of multimedia equipment connector 102, for example for PCI connection. With advantage it is possible to place into auxiliary equipment 104a place next modules and circuits, namely block 149 of communication modules which ensure data transfer by next communication protocols, which are in multimedia auxiliary equipment not available, fins data are by auxiliary equipment microprocessor 133 evaluated and hand into multimedia equipment and alternatively into next equipment. The block 149 of communication modules with advantage includes module 134 for signal transmission in the frequency band 5,5 kHz, which receives and transmit into cellular phone 100 data, which ordinary cellular phone is not able to receive, with advantage heart rate data by chest belt scanned, in addition module 135 of BT/BLC/ANT transmission by the use of protocols Bluetooth Low Energy (BLE), ANT, alternatively Bluetooth (BT) and next communication protocols with the advantage module 136 of RF transmission (band 800-900 MHz), module 137 of WiFi transmission and also module 138 of ZigBee protocol transmission. Up to date multimedia equipment, for example cellular phones with touch display, have available for multimedia equipment applications control only virtual keyboard and icons displayed on touch display, which is not I the time of multimedia equipment sleeping regime active. Touch display operation is very energy demanding that is why it is required to operate multimedia equipment in the regime of automatic sleeping, as long as it is possible. But multimedia equipment activation, which is in the sleeping regime, requires from user a few operation, what is lengthy and makes substantial trouble in the moment in which it is necessary call for help for person with loss of consciousness health problem, loss of spectacles, in darkness and similar. This problem is solved by push buttons 110 and 111 placed on auxiliary equipment 104a. The advantage over current state is prompt accessibility and identification of push buttons by touch even in dark and immediate multimedia equipment activation, even if it is in the sleep regime. Push buttons 110 and 111 activate microprocessor 133 of auxiliary equipment, which immediately activate corresponding applications in multimedia equipment regardless if is in this moment the multimedia equipment active or in the sleeping regime. The advantage of push buttons 110 and 111 using for calling for help in the case of emergency, at what tune is instantly after multimedia equipment activation send message to specified location including case that the multimedia equipment is in the energy saving sleeping regime. The advantage for fast help calling by the use of multimedia equipment is using push the buttons 110 and 111 on the auxiliary equipment with advantage labeled Panic and Reset, which in cooperation with microprocessor 113 of auxiliary equipment and its software, indicators 108 and 109 with advantage LED and acoustic signalization 112, controlled by microprocessor 133 With advantage is after the press of push button 110 by which is assigned function panic going on starting of acoustical and optical warning announcing that was activate request to send call for help by the acoustic signalization 112 and optical signalization by indicator 108 with advantage red color LED alert by blinks activation of request to call for help. During die period of which is warning signals activated it is possible to cancel the activation of request to send call for help by the use of push button 111 with advantage labeled Reset. After this the indicator 108 stops blinking and also stops warning signal of acoustic signalization. If the request to send call for help was not canceled by push button Reset, the blinking of indicator 108 is changed to permanent lighting until multimedia equipment accept request to send call tor help. Acceptance of request to send call for help foe multimedia equipment confirms, the indicator 108 stops lighting, stops acoustic signalization and for short tune is lighting indicator 109 with advantage green color LED confirms call for help acceptance. Push buttons 110 and 111 in addition reduce energy consumption because both multimedia equipment and auxiliary equipment 104a can be in the sleeping regime also in the case of probability of incident requires urgent call for help.

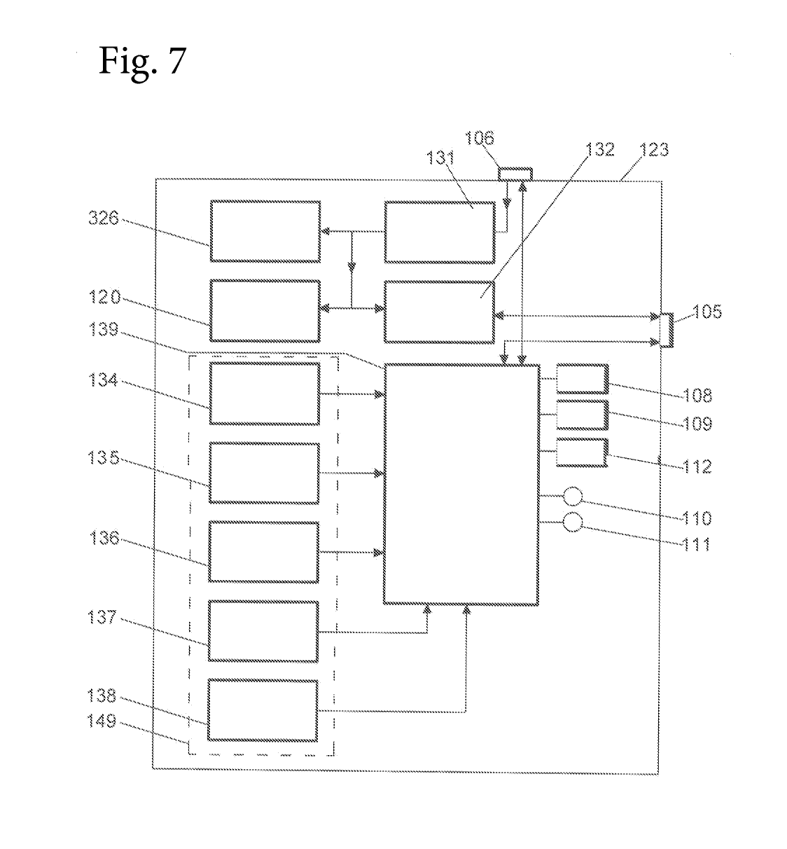

FIG. 7 Illustrates block diagram of other auxiliary equipment, on its board 123 of electronics is ordinary microprocessor 133, as presented in the FIG. 6, substituted by microprocessor 139 with two USB ports, which can at the same time master two USB connectors 102, 105 and to control data stream transmitted via these USB connectors. This facility is in the FIG. 7 used in the way that data stream of connector 102 USB of multimedia equipment after its inserting into second USB connector 105 of auxiliary equipment is by microprocessor 139. FIG. 2, directed to the next connector 106 and on the contrary, what makes possible communication of multimedia equipment via its USB connector 102 overlap by its auxiliary equipment 104 and next connector 106 with next equipment which is connected by the USB cable to the auxiliary equipment.

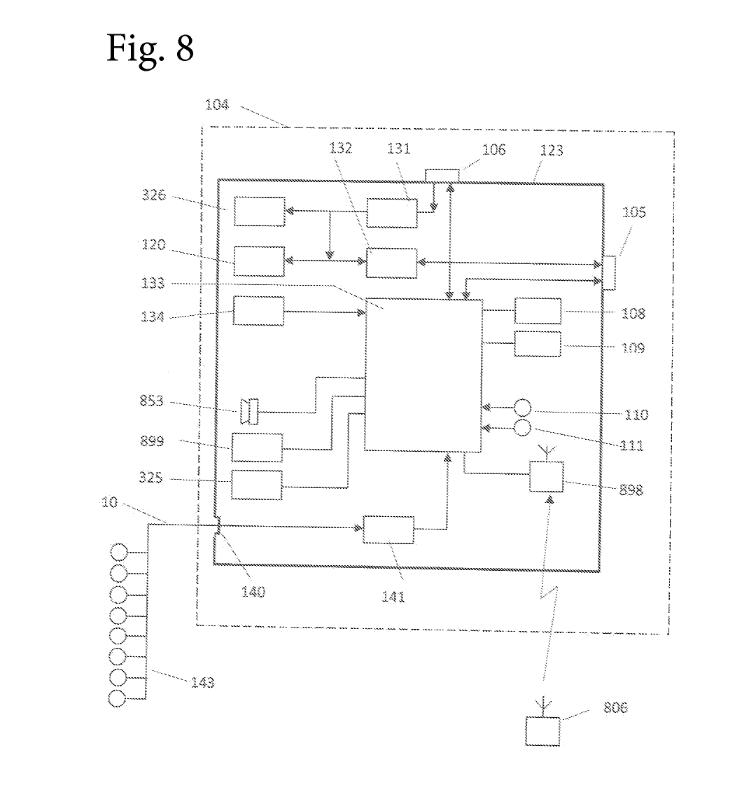

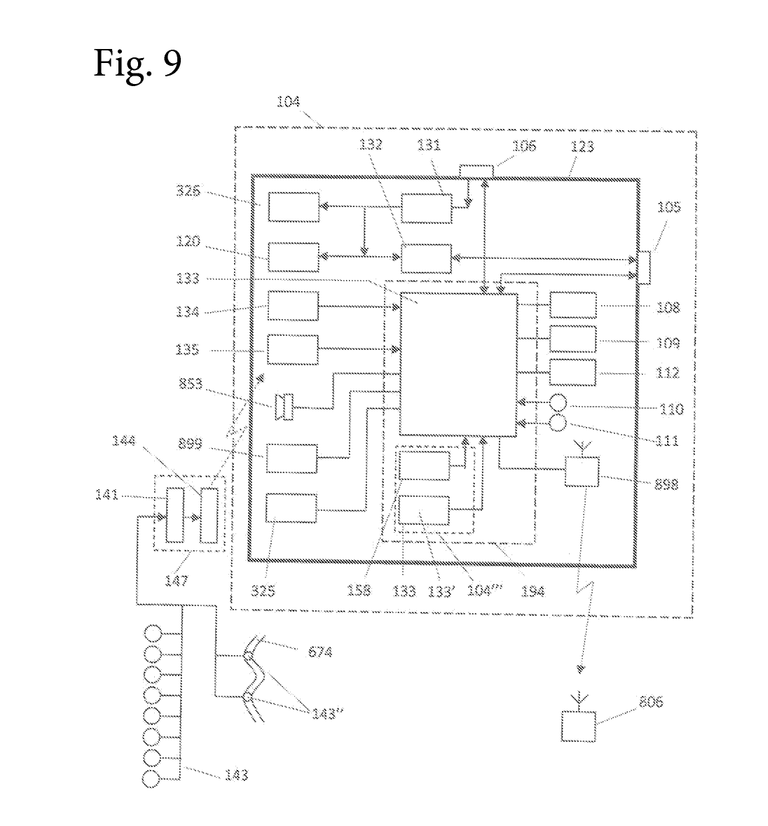

FIG. 8 illustrates block diagram auxiliary equipment 104 board 123 of electronics which is extended by the possibility of heart signals processing with advantage of EKG signals and/or heart beat and/or arrhythmia, which are on the fly evaluated and transfer for digital or graphical screening on display 325 of auxiliary equipment 104 and/or in multimedia equipment 600, where are sent via second connector 105. With advantage are data stored into memory with advantage removable memory card 899 placed inside auxiliary equipment 104 or in multimedia equipment 600, ready for relocation into suitable equipment, with advantage PC, for data transfer. By this with advantage is auxiliary equipment 104 separately or in cooperation with multimedia equipment 600 or multimedia equipment 600 separately adapted for using as “hotter” with advantage observe curves and data of heart activity on-the-fly, which are simultaneously stored into memory, what present days technology makes not possible. With advantage are data, namely heart activity, transferred continuously via communication module 898 or via multimedia equipment 600 and mobile data and/or voice net to the server 806 and from here are available to authorized subscriber with advantage medical staff or to the surveillance center. With advantage it is possible to watch if the heart activity is standard or if there are trouble, uncover active or incurred damage of heart muscle, primarily heart attack and also continuous screening of ischemic heart disease during load, and on the display of multimedia equipment 600 with advantage formed by cellular phone 100 and/or auxiliary equipment 104 are displayed EKG curve and/or with advantage at the same time waveform of heart rate and/or arrhythmia, all over long period or permanently in the conditions of standard patient activities or sports activities. Deviations from standards are announced by warning signal of acoustic converter 853 which is placed in auxiliary equipment 104 or in multimedia equipment 600 and/or on display, which is in the case that is not reset by the push button reset 111 change into alarm transmitted via communication module 898 with the advantage into server 806, available to authorized subscriber with advantage to the surveillance center. Alarm can be with advantage also activated by the push button 110 panic, which is with advantage reset able by the pushbutton reset 111. With advantage are pushbuttons reset 111 and panic 110 stand-alone buttons regardless of auxiliary equipment 104 or multimedia equipment 600 operations always ready for immediate using, what is in today's technology not possible. Electrodes 143 EKG pasted on skin of patient or with advantage placed on the chest belt 647, illustrated in FIG. 9, or in combination of both scan voltage arising by heart activity and are connected via connector 140 to auxiliary module 141 Front-End inputs, by which are signals amplified, filtered, digitized and converted into data and after that are processing by microprocessor 133 in the case that auxiliary equipment 104 is with advantage placed in pocket, small box on the belt, bangs on the chest belt or placed on bracelet, thus is adapted for conductive connection of electrodes. In case that auxiliary equipment 104 is born stand-alone and conductive connection of electrodes in not possible, then is EKG signal processed with advantage in monitor of heart signals 147 with advantage placed on chest belt 647 and/or near the waist and/or born stand-alone and resulting data are transmitted by the use of modules 135 and 144 of BT/BLC/ANT transmission and module 136 of RF transmission into auxiliary equipment 104, see FIG. 9 which shows in in full detail, where are be processed and displayed or are send to next equipment with advantage to multimedia equipment 600, the above mentioned or via communication module 898 and mobile data or voice net to the server 806. In the case of heart rate is with advantage used transmission in the frequency rate 5 kHz.

FIG. 9 illustrates alternative board 123′ of electronics block diagram arrangement with additional microprocessors of auxiliary equipment 104 for EKG signals evaluation, which are send for digital or graphical representation on display 325, which are pre-processed by monitor 147 of heart signals placed near electrodes 143 EKG outside the board 123 of electronics, with advantage on chest belt 647, in which is placed module 141 Front-End with module 144 of BT/BLC/ANT transmission with advantage BT or BEE or ANT providing wireless data transmission from module 141 Front-End to module 135 of BT/BLC/ANT transmission with advantage BT/BLE/ANT placed in auxiliary equipment 104. With advantage are modules 141 and 144 fixed on chest belt 647 in the way to be leads of electrodes 143 EKG placed on chest belt 647 or/and pasted as short as possible. The advantage of this arrangement is shortening of leads from electrodes 143 EKG and by this decrease level of noise. Electrodes 143 EKG 143′ with advantage can form part of chest belt bill or can use combination with pasted electrodes 143. By the use of auxiliary module 194 of microprocessors with advantage using one or more microprocessors placed on board 123″ of electronics or on next more auxiliary equipment 104′″ it is possible increase power and speed data processing by the use of board 123′ of electronics of auxiliary equipment. Second microprocessor 133′ and next microprocessor 133″ with advantage communicate by the use of microprocessor 133. With advantage it is possible in each microprocessor use different operating system, Android, Symbian, Windows Mobile for example and so with advantage extend sphere of data processing by the use of next general purpose operating systems, optimize abilities of auxiliary equipment with respect to given application.

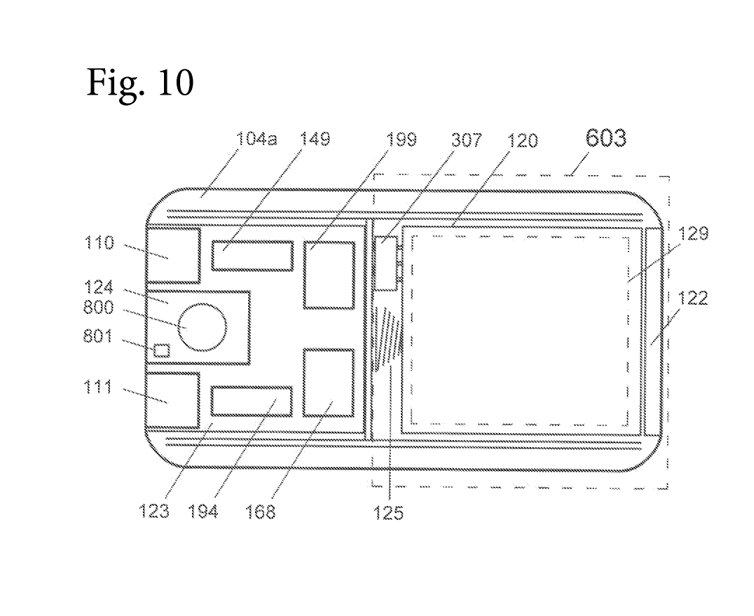

FIG. 10 illustrates a top view to mechanical arrangement of modified auxiliary equipment 104a and placement of its main parts. Auxiliary accumulator 120 is inserted into placement mechanism 603 of modified auxiliary equipment 104a after small door 122 opening and is connected by the use of contacts 307 for accumulator connection. Small door 122 after closing acts again the stress of spring 125 of accumulator sliding and providing auxiliary accumulator 120 against sliding out. Next is to the modified auxiliary equipment 104a board 123 of electronics fixed, with cut-outs which uncover lens 800 of photo camera and by it also transparent hole 124 of multimedia equipment and so its speaker 801. As illustration of electronics blocks and modules placement are on the board 123 depicted module 199 of continuous recharging of basic accumulator 129 which is placed under auxiliary accumulator 12) and recharging of auxiliary accumulator 120 from external source, next block 149 of communication modules, auxiliary module 194 of microprocessors and module 168 of auxiliary unites. With advantage are here placed two mechanical push buttons. By the push button 110 Panic it is possible to start alarm for to call for help, if this push button shell be pushed by mistake, it is possible the alarm cancel by the push button 110 reset. The advantage over equipment without mechanical buttons name and reset is, that mentioned mechanical buttons are permanently available for the case of emergency.

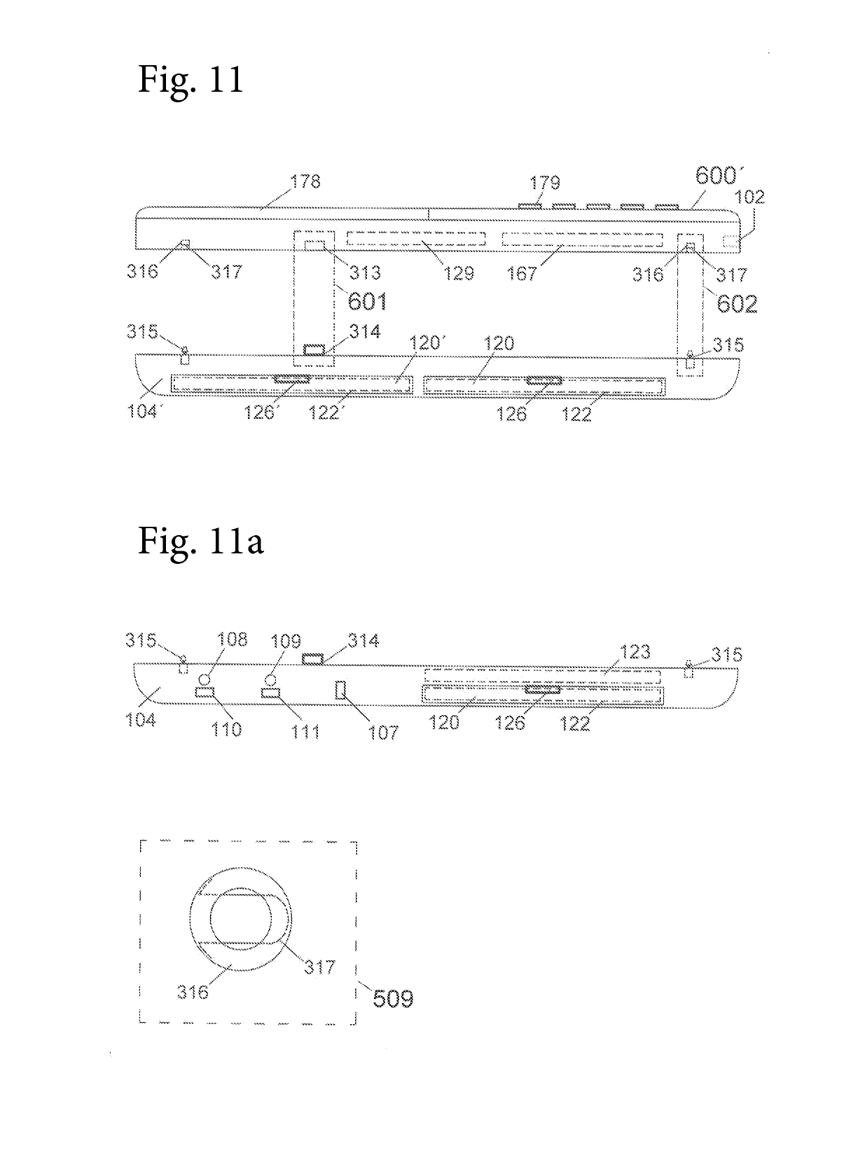

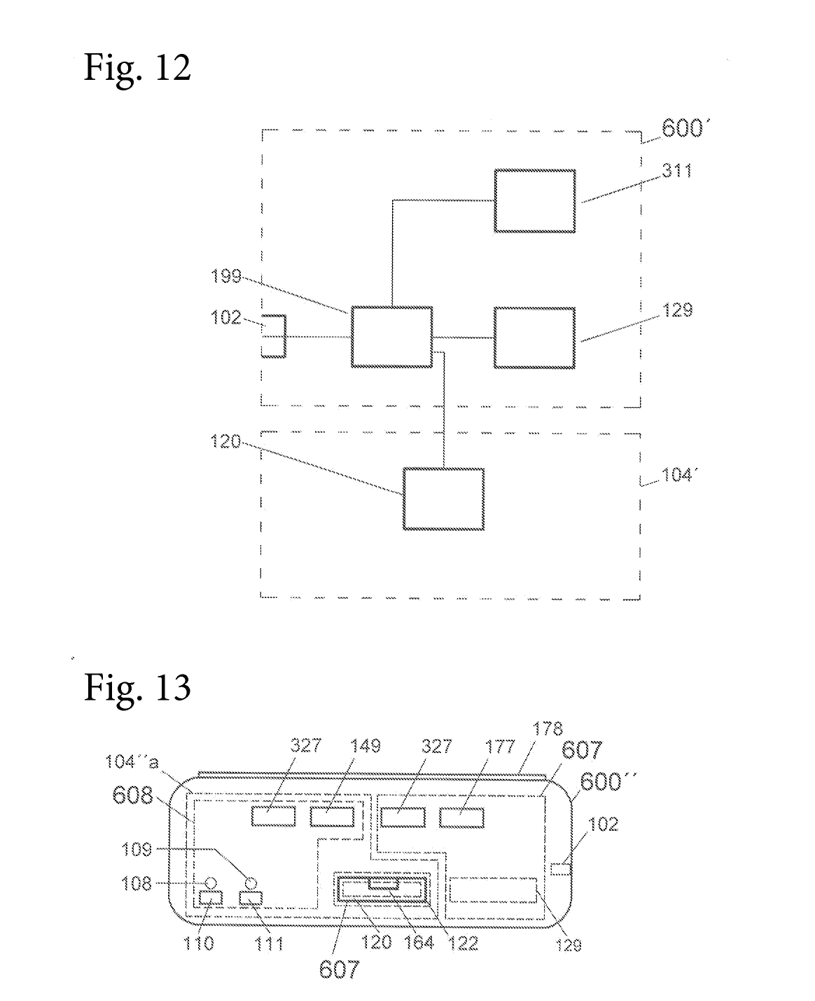

FIG. 11 illustrates a side view to modified multimedia equipment 600 with electronics 167 of multimedia equipment designed together with specific auxiliary equipment 104′ for easy removing mechanical and electrical connection/disconnection one or more specific auxiliary equipment. Mutual mechanical connection is made by the use of fixing elements 602 designed for this purpose and placed on multimedia equipment. With advantage are in this case on the auxiliary equipment placed dowel pins 315, which fit in contoured hollow 316 with transverse spring 317 placed on multimedia equipment. By this mechanical connection are both equipment mutual fixed so that during manipulations by multimedia equipment its position as well as position of all units and parts in it is assured again random moving. Electrical connection of multimedia equipment and auxiliary equipment is by the connecting elements 601 with advantage formed by linking connector 313 of multimedia equipment and its counterpart, what is linking connector 314 of auxiliary equipment. Connector 102 USB of multimedia equipment is not used for mutual electrical connection multimedia equipment and auxiliary equipment, is not covered and that is why it can be used by multimedia equipment. Auxiliary equipment is with advantage possible mechanical and electric connect to multimedia equipment in the need expand multimedia equipment function. Auxiliary equipment include auxiliary electric and electronic units or parts and with advantage control elements. For example, auxiliary equipment 104′ in FIG. 11 includes auxiliary accumulator only, namely auxiliary accumulator 120 placed behind small door 122, block diagram see FIG. 12, and next auxiliary accumulator 120′ placed behind next small door 122′, secured by lock 126′ of small door. To the contrary variation of auxiliary equipment 104′ in FIG. 11a includes in addition board 123 of electronics which performs next functions, for example see FIG. 6 up to FIG. 9. On auxiliary equipment there are apart from small door 122 for auxiliary accumulator 120 inserting, more placed control elements, with advantage push buttons 110 panic and 111 reset, switch 107, first indicator 108 and second indicator 109. In the case that auxiliary equipment is not used, auxiliary equipment is substitute by cover. Detail 509 illustrate example of hollow 316 of specific auxiliary equipment 104′ of fixing elements 602 which is formed by dowel pin 315 solidly connected to multimedia equipment 600, which fit in contoured hollow 316 with transverse spring 317, solidly connected to modified multimedia equipment. After dowel pin 315 inserting into hollow 316 transverse spring 317 fit in groove/of dowel pin 315 what cause connection fixing. This arrangement can be with advantage used also for additional lugs installation on multimedia equipment after its manufacture.

FIG. 12 illustrates block diagram of continuous charging of the basic accumulator 129 of multimedia equipment 600′ (see FIG. 11) and auxiliary accumulator 120 placed in connected auxiliary equipment 104′. In this case is auxiliary equipment linked up mechanical and electrical to multimedia equipment and that is why circuits are simple. Module 199 of continuous basic accumulator 129 is charging in this case recharge accumulators 120 and 129 depending on its need and in the same time supply electric circuits 311 of multimedia equipment. From external source via connector 120 of multimedia equipment 600′ are at the same time recharged auxiliary accumulator 120 and basic accumulator 129. After the external source disconnection from connector 102 module 199 continuously recharge basic accumulator 129 of multimedia equipment 600 using energy of auxiliary accumulator 120.

FIG. 13 illustrates example of auxiliary equipment 104″a which is integrated into compact multimedia equipment 600″. The compact multimedia equipment 600″ consists of block 607 of multimedia equipment and integrated auxiliary equipment 104″a, which includes block 608 of auxiliary equipment electronics, auxiliary accumulator 120 placed in placement mechanism 603, which makes possible to change accumulator 120 by the use of small door 122 provided by lock 164, and display 178 of multimedia equipment. Block 608 of auxiliary equipment electronics with advantage includes push buttons 110 and 111, indicators 108 and 109, microprocessor unit 158 and block 149 of communication modules. Block 607 of multimedia equipment consists of SIM card 177, microprocessors unit 327 of multimedia equipment and basic accumulator 129. The function of auxiliary equipment 109 integrated into compact multimedia equipment 600″ are the same as function depicted sooner, with advantage provided easy due to replaceable auxiliary accumulator 120, which supply compact multimedia equipment 600″ and at the same time recharge basic accumulator 129, more block 149 of communication modules which makes possible communication by the use of different media, microprocessor unit 158 of auxiliary equipment, which communicate with microprocessors unit 327 of multimedia equipment and with advantage is using different operating system than microprocessors unit 327 and by this makes possible communication and another modules which assure mentioned function.

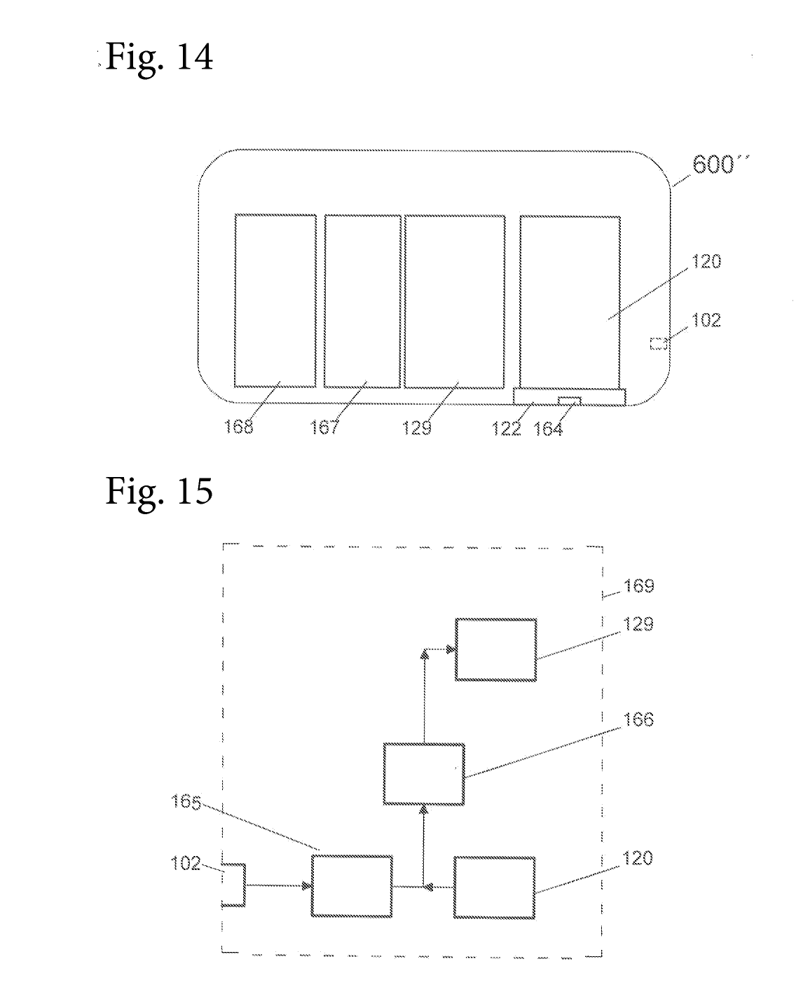

FIG. 14 illustrates arrangement of compact multimedia equipment 600″ main pans alternative supplied from auxiliary accumulator 120. There are depicted basic accumulator 129 and auxiliary accumulator 120, electronics 167 of multimedia equipment and module 168 of auxiliary unites of multimedia equipment.

FIG. 15 illustrates block diagram of module 169 of recharging electronics placed on the electronics 167 of compact multimedia equipment 600″, see FIG. 14. Module 169 of recharging electronics provides continuous recharging of basic accumulator 129 of compact multimedia equipment 600″ (see FIG. 14) from auxiliary accumulator 120. The auxiliary accumulator 120 charging from external source connected to the connector 102 is going via charger 165 of multimedia equipment and at the same tune is charged basic accumulator 129 via charger 166 with converter. After the external source disconnection is basic accumulator 129 continuously recharged from the auxiliary accumulator 120, just as via charger 166 with converter, which rise up voltage taken from auxiliary accumulator 120 to value needed for proper recharging of basic accumulator 129 at first and control charging.

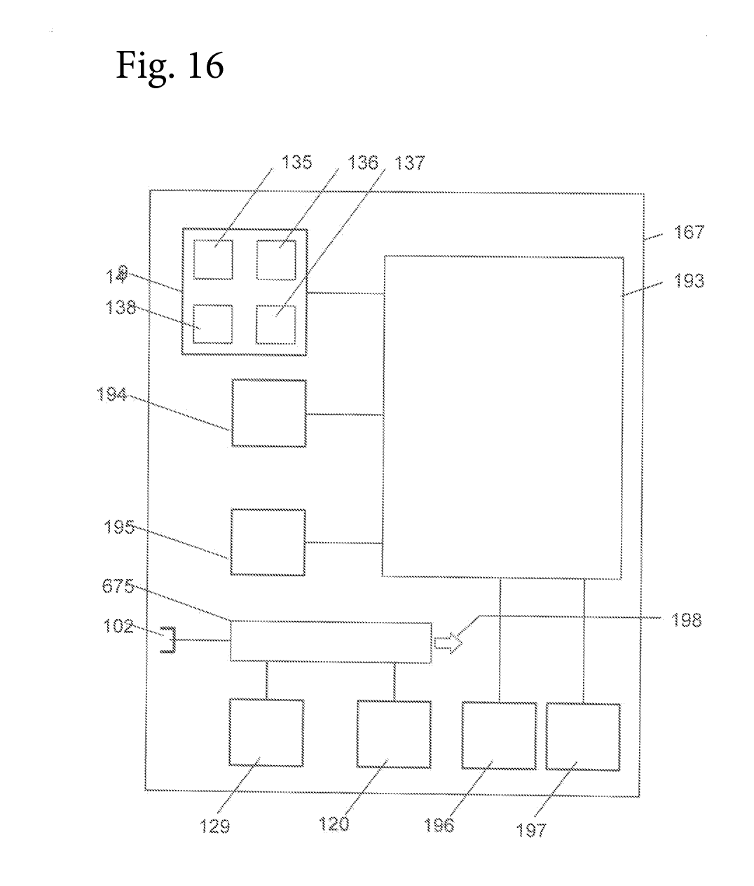

FIG. 16 illustrates block diagram of modules placed on electronics 167 of multimedia equipment of compact multimedia equipment 600″, see FIG. 14 above. To the operational module 193 of auxiliary equipment axe connected to module 196 of display, key board module 197, block 149 of communication modules with advantage including module 135 of BT/BLC/ANT transmission, module 136 of RF transmission, module 137 of WiFi transmission, module 138 of ZigBee transmission and auxiliary module 195 of multimedia equipment. Basic accumulator 129 and auxiliary accumulator 120 are connected to electronics 675 of accumulators charging, which aside from recharging of both accumulators provide also feeding circuits of multimedia equipment, what arrow 198 depicts.

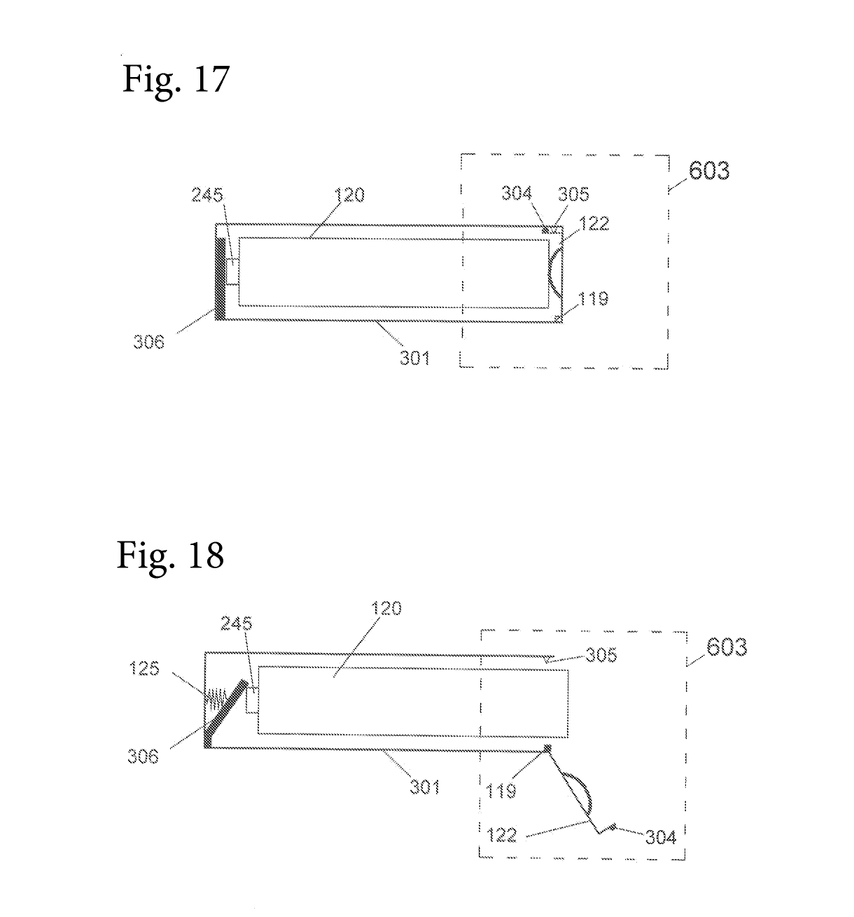

FIG. 17 illustrates example of simple placement mechanism 603 of auxiliary accumulator 120. Casing 301 define space for auxiliary accumulator 120 placing with bronze spring 306 and contacts 245 of accumulator inside. After inserting is accumulator provided by the small door 122 turning on binge 119 with latch 304 and overhang 305.

FIG. 18 illustrates how after small door 122 opening spring 125 together with bronze spring 306, which provide also connection to contact 245 of accumulator 120, slid out accumulator 126 partly from casing 301, so that it is possible to manipulate with accumulator by hand.

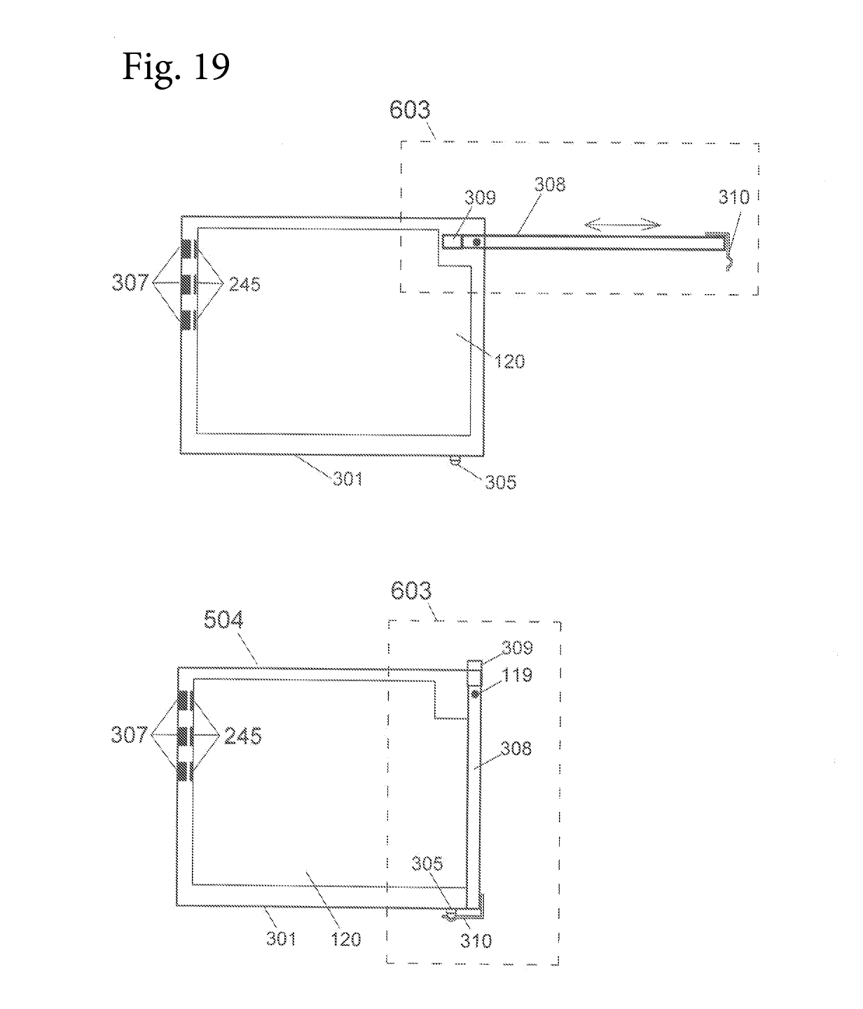

FIG. 19 illustrates other variant of placement mechanism 603. Auxiliary accumulator 120 is after inserting into casing 301 of auxiliary equipment provided by small lever 308. Detail 504 illustrates accumulator 120 providing by small lever 308 in closed position, accumulator 120 is pressed to flexible contacts 307 for accumulator connection in the easing 301 by contacts 245 of accumulator and is provided in the cover by the end of small lever 308 turning on hinge 119 reclining cut-out in side of casing 301. Small level 308 is provided in the reclined position by the spring 310, which click into overhang 305 on casing 301. During accumulator exchange is small level 308 turn 90° and by the small level 308 pulling slide out. By the advantage can be in this way inserted recharged accumulator.

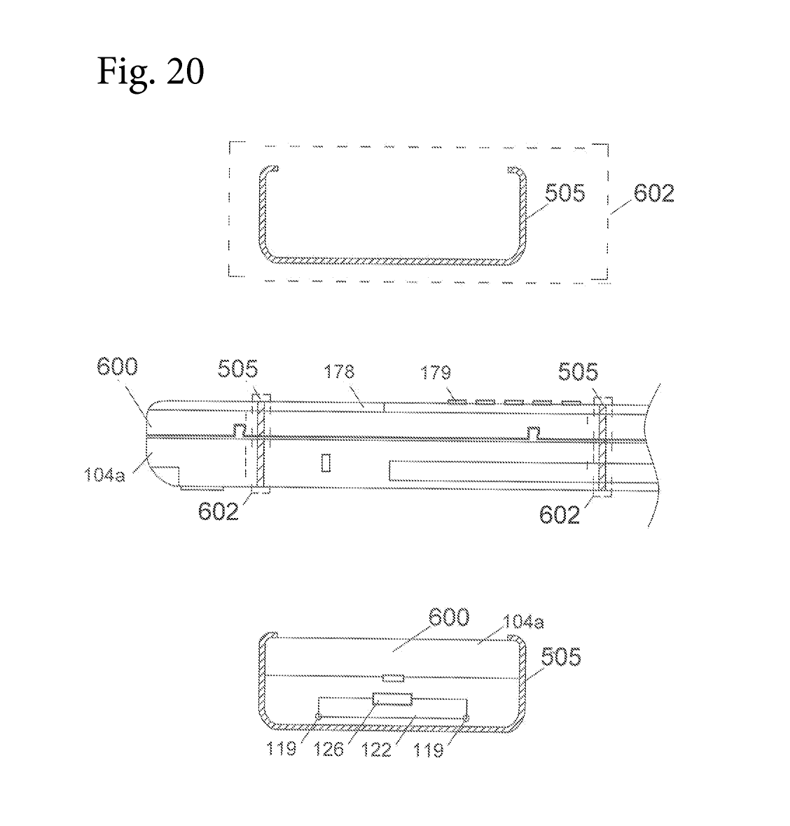

FIG. 20 illustrates mechanical connection of unmodified multimedia equipment 600 and modified auxiliary equipment 104a by the use of fixing element 602 formed by clips 505 pulls over the both equipment.

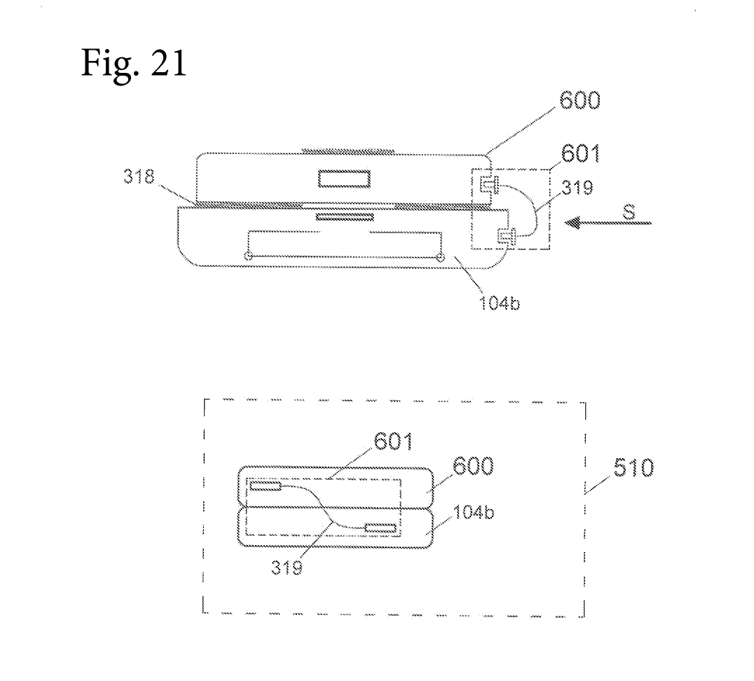

FIG. 21 illustrates mechanical connection of unmodified multimedia equipment 600 and unmodified auxiliary equipment 104b by the use of fixing element 602 formed by coupling layer 118 which can be Velcro fastening or paste layer. Electrical connection both equipment provides connecting elements 601 formed by linking small cable 319 and two its connectors. Detail 510 in FIG. 21 illustrate view in S direction to connecting elements 601 formed by small cable 319 with connectors, which makes possible electrical connection of auxiliary equipment and auxiliary equipment in the case that linked connectors are not above.

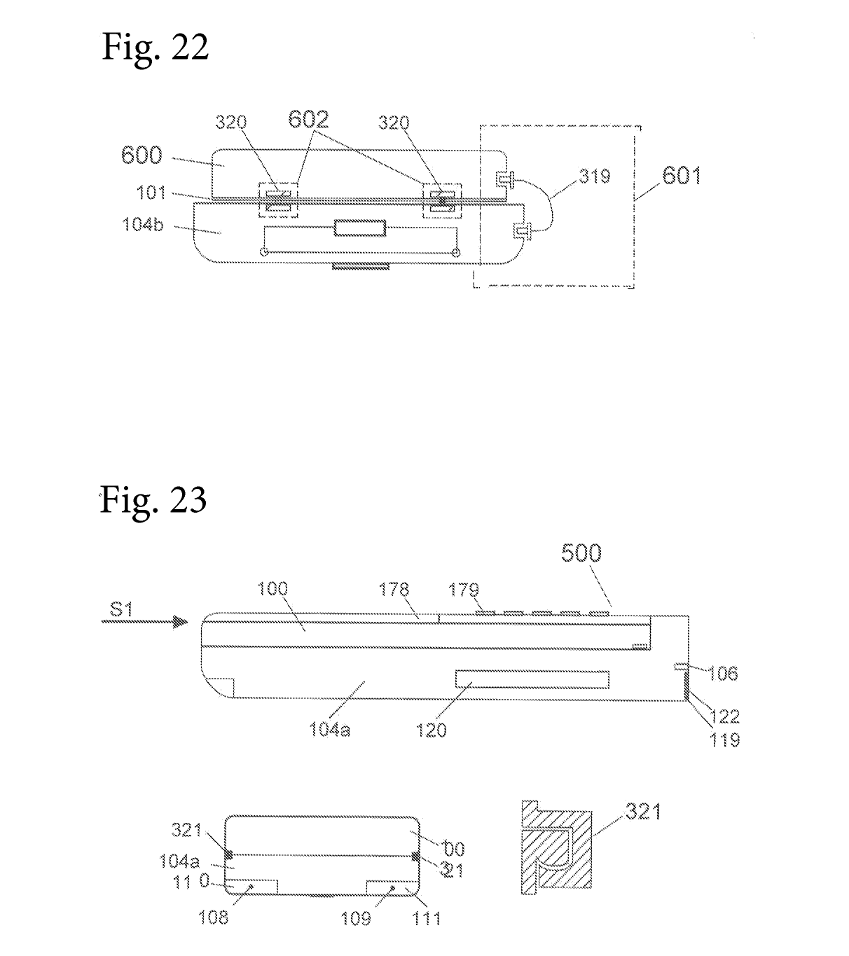

FIG. 22 illustrates mechanical connection of unmodified multimedia equipment 600 and unmodified auxiliary equipment 104b by the use of fixing element 602 formed by mechanical coupling elements 320, with dowel pins 315 and cut through hollow 316 with transverse spring 317, FIG. 11 above. Electrical connection is by the use of connecting elements 601 formed by linking small cable 319 with two connectors.

FIG. 23 illustrates mechanical connection of multimedia equipment formed by modified cellular phone 100 and modified auxiliary equipment 104a by inserting grooves of multimedia equipment into grooves of auxiliary equipment, what form fixed but separable connection, see Detail 321.

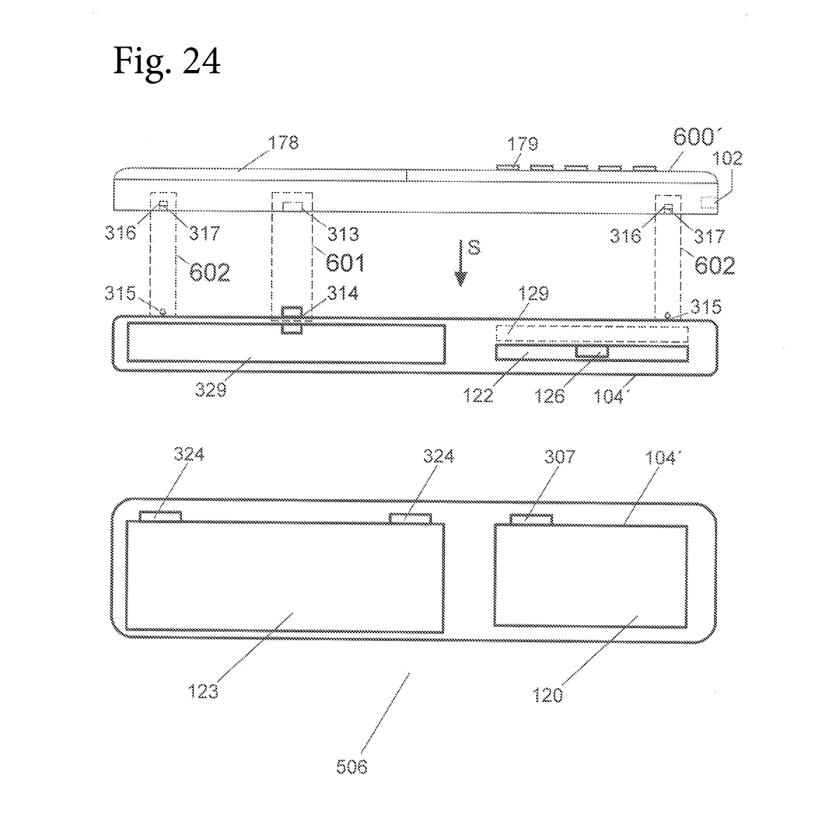

FIG. 24 illustrates a view to mechanical arrangement of specific auxiliary equipment 104′ created in bum of carry case which is mechanical connected to modified multimedia equipment 600′ by the use of fixing element 602 formed by dowel pins 315, which are after fitted in contoured hollow 316 with transverse spring 317 by this spring fixed. Electric connection is provided by connecting elements 601 formed by connectors 313 and 314. Auxiliary equipment board 123 of electronics with circuits extending function of multimedia equipment 600′ is by the use of small door 329 inserted into one or more mutual connected elements 324. Auxiliary accumulator 120 is inserted by the use of small door 122. Detail 506 illustrates example of auxiliary equipment board 123 of electronics and basic accumulator 129, 120 in placing specific auxiliary equipment 104′. Basic accumulator 129 is fixed the basic accumulator 129 above.

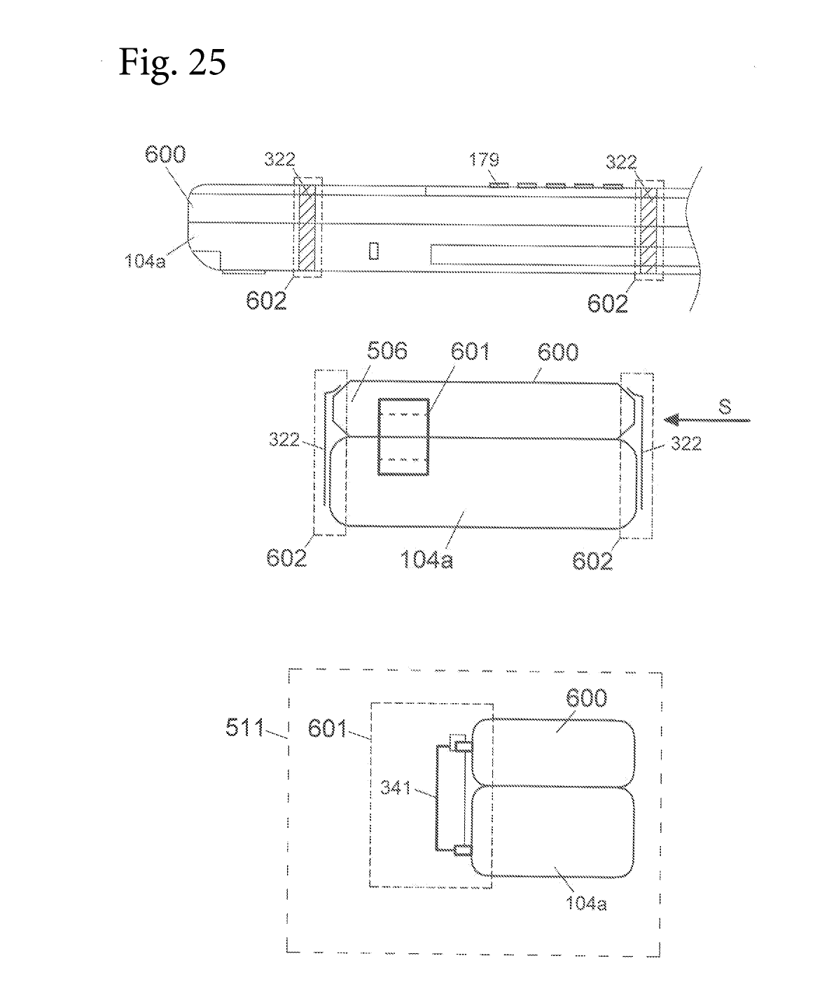

FIG. 25 illustrates a view to mechanical arrangement of modified auxiliary equipment 104a which is mechanical connected to unmodified multimedia equipment 600′ by the use of fixing dement 602 formed by flexible lugs 322, which arc pasted or in a different way fixed by all or partial its surface to modified auxiliary equipment 104a. Ends of flexible lugs are contoured so they fit tightly to edge of unmodified multimedia equipment 600′. This removable connection makes possible quick mechanical connection of both equipment by the press them into flexible lugs 322. In this case both multimedia equipment and auxiliary equipment have they connectors above and therefore it is possible both connectors electrical link up by connecting elements 601 formed by U-connector 341, detail 511.

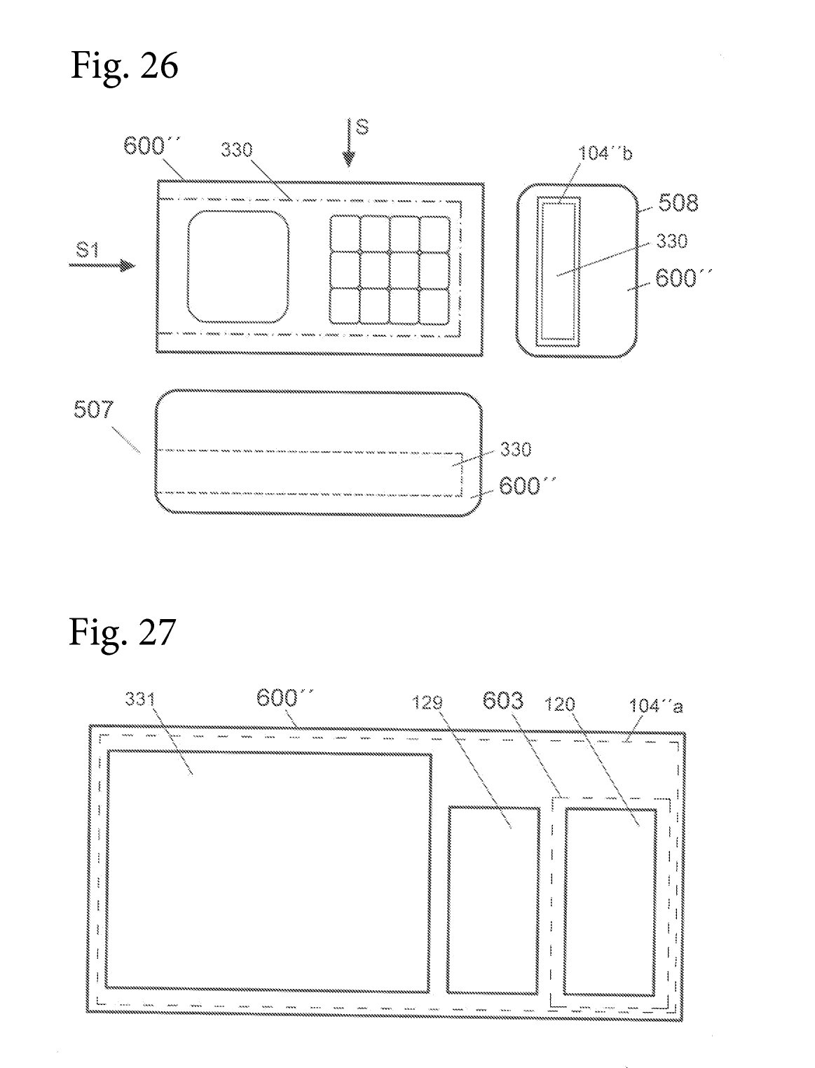

FIG. 26 illustrates a view to mechanical arrangement of removable auxiliary equipment 104″b which is mechanical connected to compact multimedia equipment 600′ by inserting into space 330 formed multimedia equipment inside (connectors not illustrated). Detail 507 illustrates view to compact multimedia equipment 600″ in direction S, space 330 for auxiliary equipment insertion is depicted. Detail 508 illustrates view in direction S1.

FIG. 27 illustrates configuration of compact multimedia equipment 600″ with advantage with extended function. In this case integrated auxiliary equipment 104″a forms integral part of the multimedia equipment. In this arrangement are modules of multimedia equipment not removable placed on common board 331 of electronics of compact multimedia equipment 600″ with functions extended. Easy removable is only auxiliary accumulator 120 from placement mechanism 603 and replaceable by charged auxiliary accumulator 120 without multimedia equipment operation interruption. Multimedia equipment is in this case formed, by cellular phone and with advantage without next function extension excluding function of easy replaceable auxiliary accumulator 120.

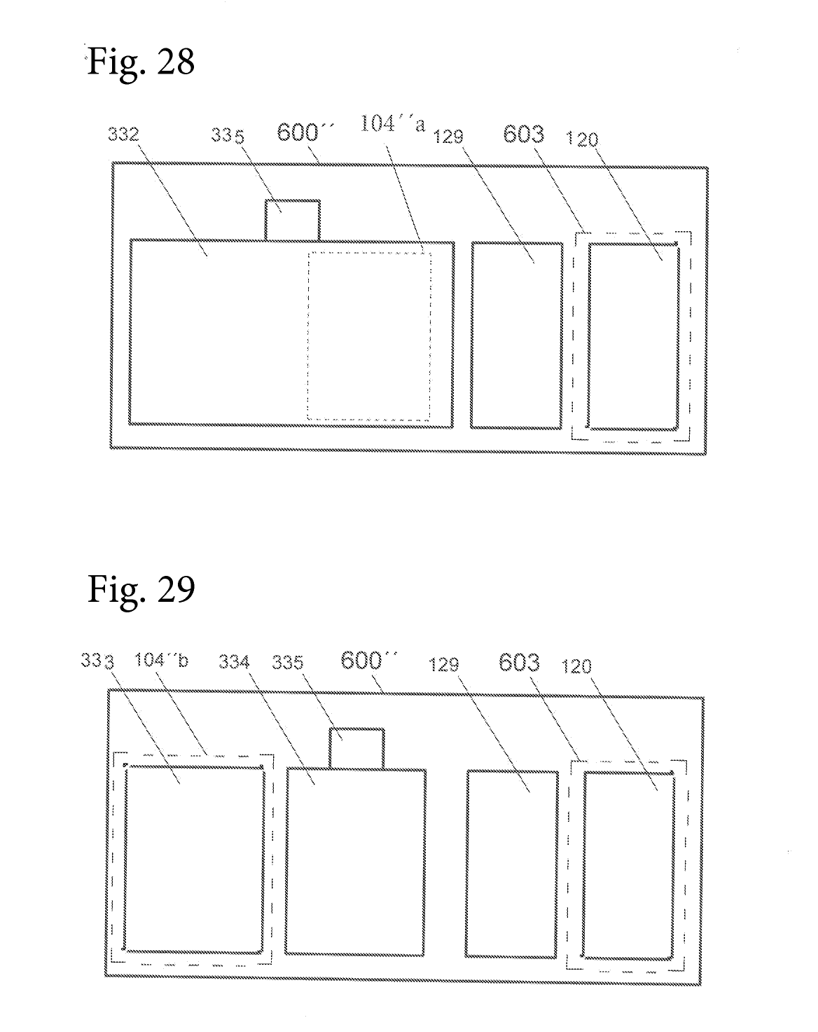

FIG. 28 illustrates another configuration of compact multimedia equipment 600″ with extended function. Integrated auxiliary equipment 104″ is in this case also integrated with multimedia equipment on common board 332 of electronics, which is replaceable in the whole by the use of linking connector 335.

FIG. 29 illustrates next configuration of compact multimedia equipment 600″ with extendable junction, where removable auxiliary equipment 104″b is placed in multimedia equipment on separate removable board 334, connected by inking connector 335. Electronics of multimedia equipment is placed on not removable on the board 333. Auxiliary accumulator 120 is easy removable from placement mechanism 603 of accumulators.

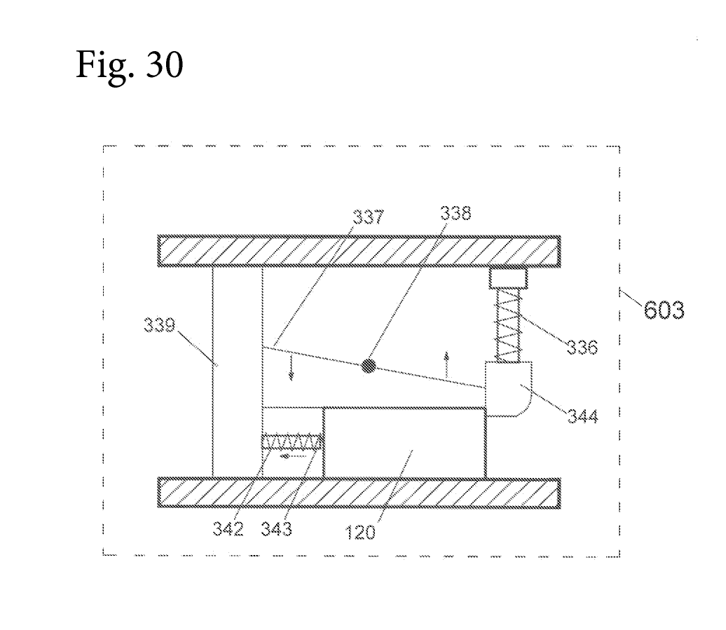

FIG. 30 illustrates operational view to placement mechanism 603 of accumulators press reacted. Auxiliary accumulator 120 insertion overcome the press of spring 336 of latch 344 and the latch 344 is lift up. Contact of auxiliary accumulator 120 sit down on corresponding contact of release mechanism 339 of accumulators and is provided by latch 344 pressed down by spring 336. Auxiliary accumulator 120 taking out starts by the auxiliary accumulator 120 pressing what press pin 343 into release mechanism 339 of accumulators and after that overturns of level 337 revolving around the pin 338. The latch 344 release makes possible to be auxiliary accumulator 120 slide out by the spring 342. Delay element, the part of placement mechanism 603 of accumulators, causes that spring 336 presses down latch 344 after some time interval, which provide reliable sliding out of auxiliary accumulator 120.

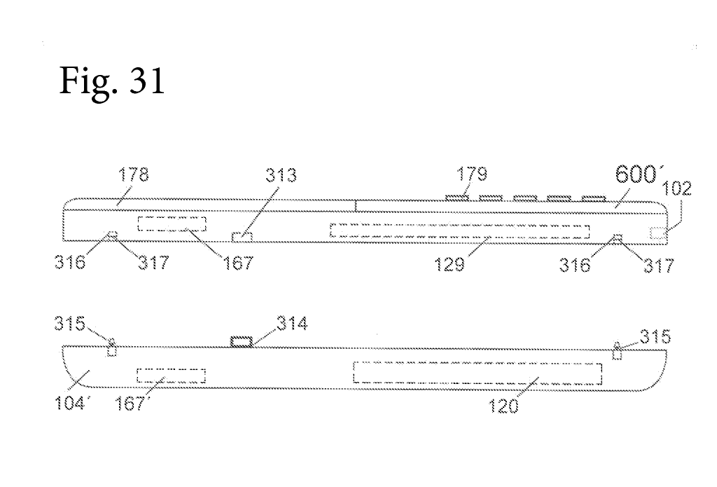

FIG. 31 illustrates next variant of modified multimedia equipment 600′ designed together with the specific auxiliary equipment 104′, see FIG. 11 above. In this case is auxiliary accumulator 120 placed the specific auxiliary equipment 104′ together with electronics 167 for its charging inside is used for continuous recharging of baste accumulator 129. As long as modified multimedia equipment 600′ operates without specific auxiliary equipment 104′, which is substitute by cover, its basic accumulator 129 is recharged from external source by electronics 167 of multimedia equipment.

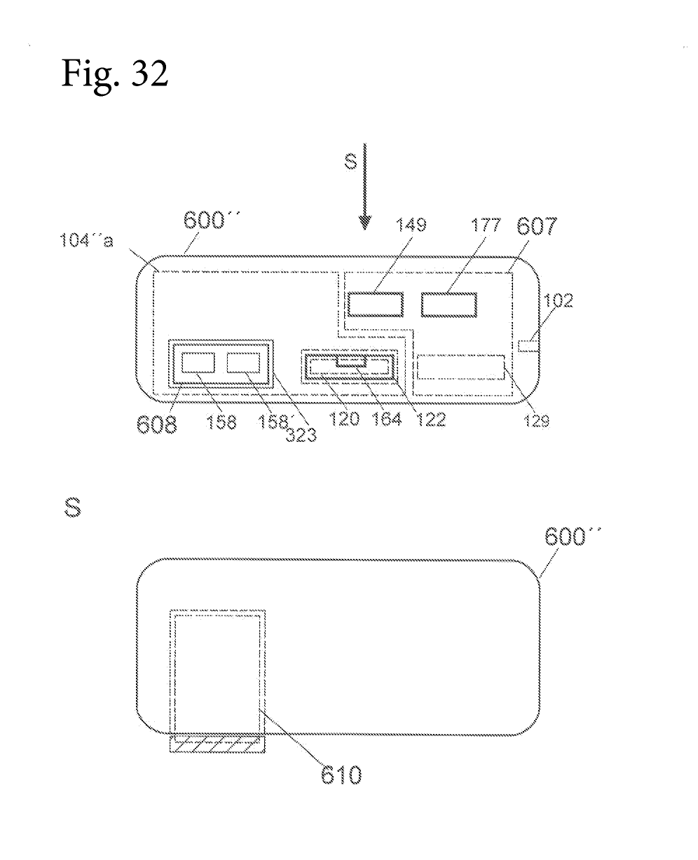

FIG. 32 illustrates next configuration of compact multimedia equipment 600″ where integrated auxiliary equipment 104′a includes mechanism 323 of block 608 of electronics placing into compact multimedia equipment 600″. In the block 608 of electronics there are microprocessors units 158 and 158′ of auxiliary equipment. Next part of integrated auxiliary equipment 104″a forms auxiliary accumulator 120 inserted through small door 122, by lock 164 provided. Cassette 610 is inserted into mechanism 323 of block placing, which can be easy and promptly changed, with advantage include auxiliary mechanical, electrical and electronic modules or parts, which extent functions of compact multimedia equipment 600″.

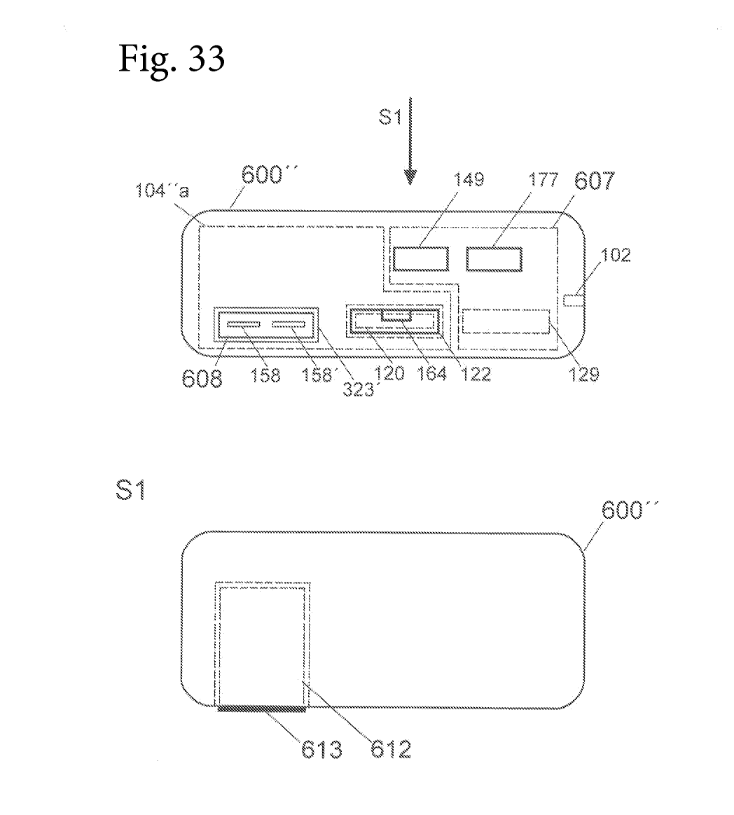

FIG. 33 illustrates variant of above FIG. 32, but simple mechanism 323′ of board 612 with panel 613 inserting is used. It is useful for simple applications in which are limited possibilities of board 612 sufficient.

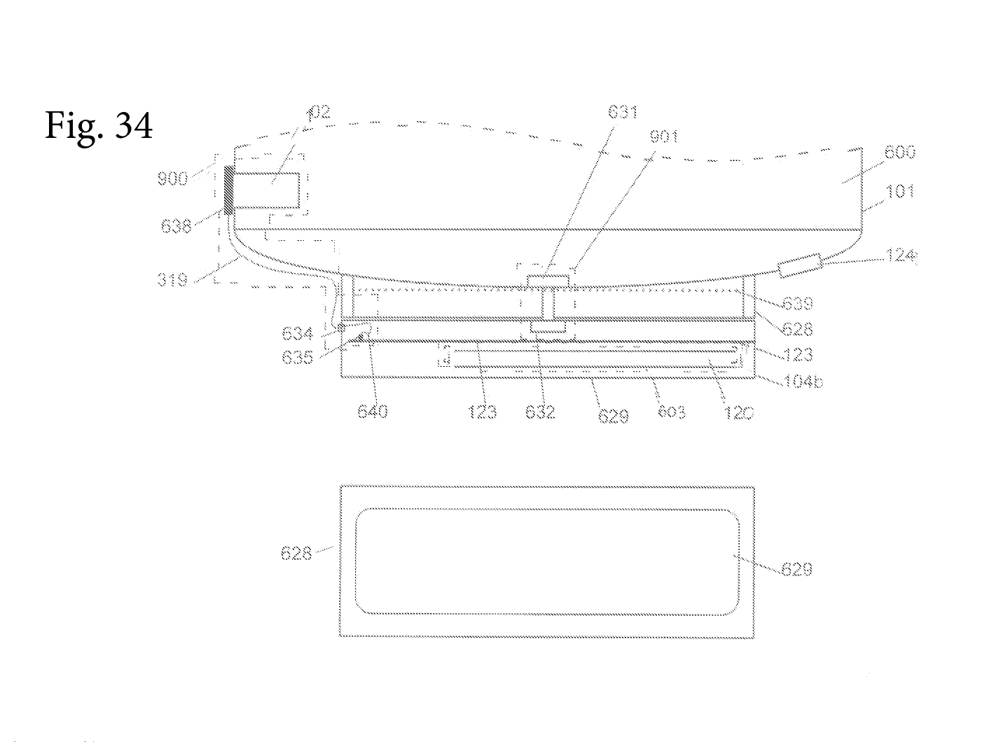

FIG. 34 illustrates way of connection unmodified auxiliary equipment 104b with bumpy cover 101 of unmodified multimedia equipment 600, where transparent hole 124 of multimedia equipment camera stay not covered. The bumpy aligns elastic pad 628 fitted tightly to bumpy cover 101 surface. The elastic pad 628 is with advantage without filling, in the case of not bumpy cover 101 it is possible use thin elastic pad 620 or omit it. Unmodified auxiliary equipment 104b is connected to cover 101 of multimedia equipment by the fastening part 901 with advantage formed by screw 631 or more screws and nuts 632 fastened to auxiliary equipment. The elastic pad 628 can be also pasted both to unmodified auxiliary equipment 104b and TO cover 101. Then advantage is that covers of multimedia equipment 600 are easy available as spare parts, so that original cover 101 can be used to restore multimedia equipment 600 to its original state and for adaptation use the spare part. Connection between connector 102, with advantage USB female, of multimedia equipment and auxiliary equipment 104b is done by link up part 900 with advantage formed by cable 319, which is one side soldered to connector 102 male soldered to PC hoard 638, by which is shorter length of cable set of connector 102 with soldered cable 319 compared to current sets. Cable 319 is with advantage pasted to cover 101, by which there is space for connector 102, and pass through the rubber part 634 in the hole into auxiliary equipment 104b, where is cable 319 soldered to solder point 635 on auxiliary equipment 104b board 123 of electronics. This makes possible to set the proper length of cable 319 from auxiliary equipment 104b to USB connector 102 different sixes of multimedia equipment. After tighten of screw 631 reach foe auxiliary equipment 104b level 639 from the level before screw 631 was tighten, in which is in the FIG. 34 depicted. Auxiliary accumulator 120 is placed in placement mechanism 603, with advantage removable during operation of multimedia equipment.

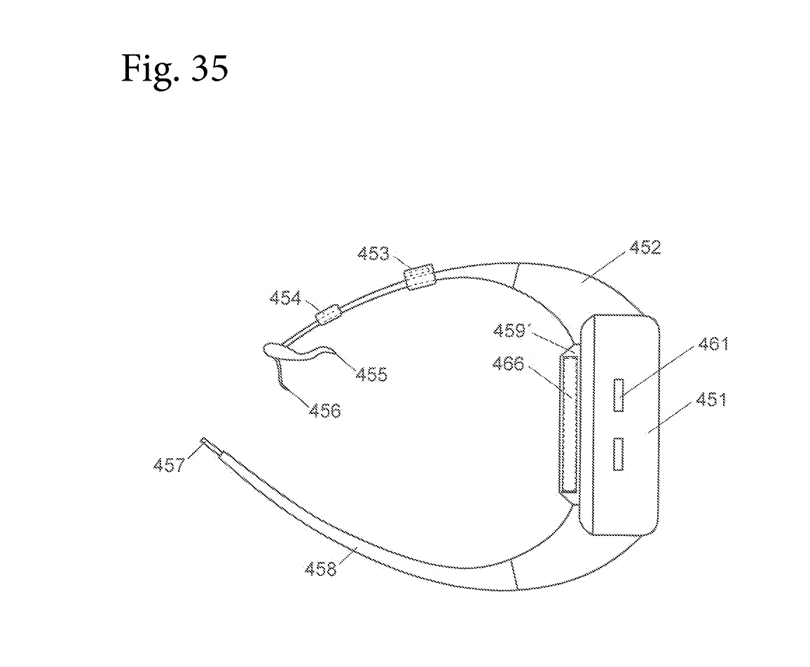

FIG. 35 illustrates cellular phone 451 in bracelet with control elements 461 with advantage of extension of abilities by the use of specific auxiliary equipment 104′ which consists of basic accumulator 129, auxiliary accumulator 120 and auxiliary board 449 of electronics placed in cover 459′ which substitute original cover of modified cellular phone 451 in bracelet or unmodified cellular phone in bracelet, how will be presented later. In both cases is cover with auxiliary equipment 104 removable and with advantage substituted by original cover 460, if it is not used. Cellular phone 451 in bracelet is fixed to the bracelet which is formed from two parts, the top part 452 of bracelet with buckle and bottom pan 458 of bracelet, which is with advantage ended by male plug 457 USB, which is bottom part 458 inside connected to bottom part 458 of bracelet. During fastening the bottom part 438 of bracelet pass through buckle 455 and is provided by pin 456. Male connector 457 USB is during fastening of bracelet inserted into half-closed belt loop 453 with connector inside forming also cover of connector 457. Replaceable accumulator is easy removable through small door 466. Belt loop 454 us used for bottom part 458 of bracelet attach.

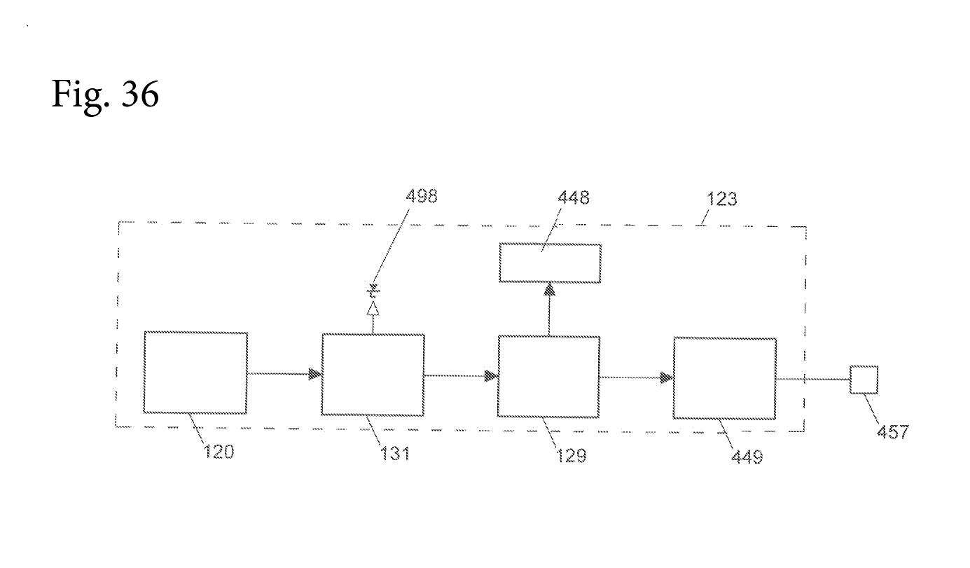

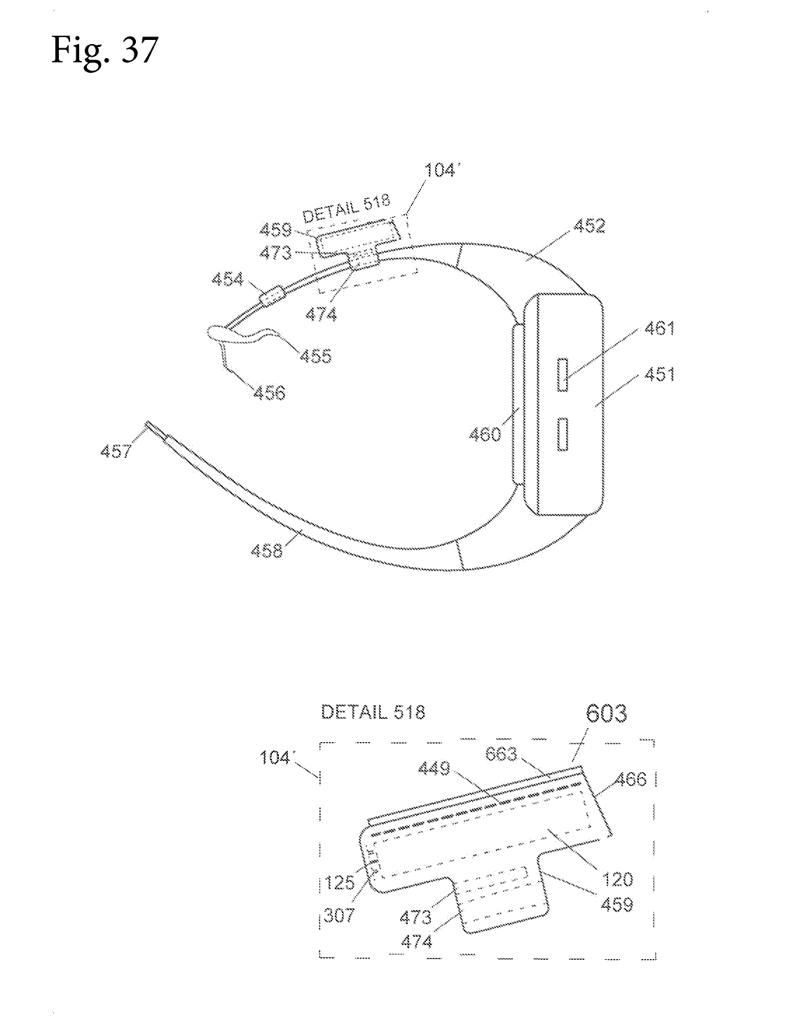

FIG. 36 illustrates block diagram of cellular phone 451 in bracelet, see FIG. 35 with the possibility of extension by the use of auxiliary accumulator 130 and auxiliary equipment board 123 of electronics. This arrangement of of cellular phone in bracelet solved the problem of limited time of operations from recharged auxiliary accumulator 120, because the recharging of its basic accumulator during walking is nearly impossible. Solving of this problem, consists of the auxiliary accumulator 120 using. Auxiliary accumulator 120 is placed and fixed in way, that it is possible quickly and easy change the discharged auxiliary accumulator 120 and recharged one which can be daily earned in the pocket, for example. Electric energy of accumulator 120 is used by charger 131 of continuous recharging of basic accumulator 129, which supply electronic 448 of cellular phone 451 in bracelet included case that recharged due to auxiliary accumulator 120 changing. Discharging of auxiliary accumulator 120 and thus its voltage decrease under minimal value necessary for basic accumulator 129 charging and therefore necessity of auxiliary accumulator 120 changing is signalized by the LED 498 blinking. Remains the possibility of the auxiliary accumulator 120 changing from external charger, which is connected to the male plug 457 USB. Auxiliary board 449 of electronics with advantage can cover next circuits for cellular phone 451 in bracelet function extension.

FIG. 37 illustrates extension of possibilities of cellular phone 451 in bracelet function, with advantage unmodified by the use of specific auxiliary equipment 104′ placed in case 459 designed for on bracelet fastening, which prolongs operation time of cellular phone 451 in bracelet and can provide next functions. Cellular phone 45.1 in bracelet is depicted with fixed case 459 of auxiliary equipment 104′ and the original cover 460. The bracelet of cellular phone 451 forms two parts, the top part 452 of bracelet with buckle and bottom part 458 of bracelet. Bottom part 458 of bracelet is ended by male plug 457. USB, which is bottom part 458 inside connected to electronics of cellular phone 451 inside, if cellular phone 451 in bracelet is not on the hand, is the ping 457 USB can be inserted into USB connector of external charger or into USB PC connector also for connection to external PC or the another multimedia equipment. On fastening on band is bottom part 458 of bracelet with fixed case 459 is connector 457 USB inserted into connector 473 of case 459, by this is electronics of cellular phone 451 in bracelet connected to electronic of case 459 and the bracelet is provided by pin 456. Detail 518 depicts placement mechanism 603 of accumulator with advantage auxiliary accumulator 120 taking out through small door 466 of case 459, by which is provided against slide out. Auxiliary accumulator 120 is easy replaceable by the use of small door 466 in cover 459 with advantage made of polyurethane or similar flexible material. This makes possible that small door 466 can form mechanical complex with case 459, because hinge of small door is replaced by thinner side of case 459. Next is depicted auxiliary board 449 of electronics, display 663, position of hole 474 for bracelet pass through case 459 and placement of USB connector 473 of cover, which is inside case 459 connected to board of electronics. Advantage is easy mechanical and electrical connection/disconnection of auxiliary equipment electronics and electronics of cellular phone 451 in bracelet without its modifications.

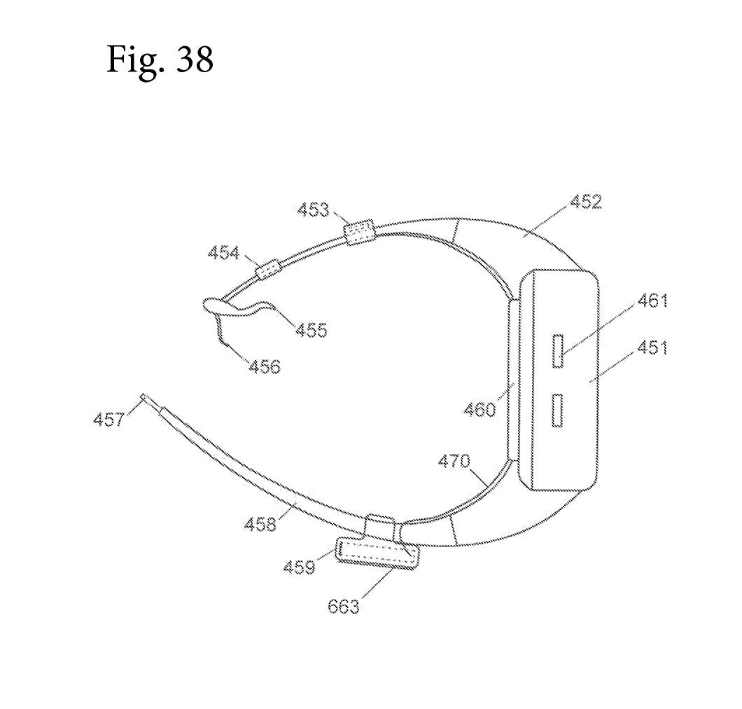

FIG. 38 illustrates variant of case 459 of auxiliary equipment fixing to opposite side of bracelet contrary to FIG. 37 in the way that display 663 is heading to user. This can be also done by swan the bottom part 458 of bracelet and top part 452 of bracelet, sec FIG. 37, in the case of modified cellular phone in bracelet. Without swapping it is necessary use for connection, to electronics of cellular phone 451 in bracelet use auxiliary cable 470 going from belt loop with connector 453, which is to the bracelet pasted or fixed in another way.

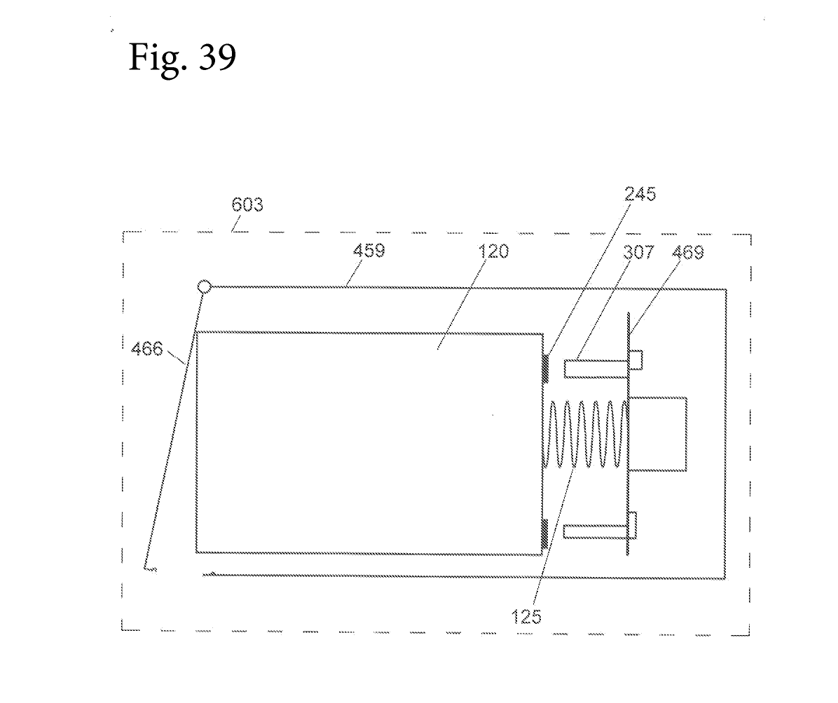

FIG. 39 illustrates other variant placement mechanism 603 of auxiliary accumulator 120 with advantage example of auxiliary accumulator 120 and board 448 of electronic recharging control of cellular phone 451 in bracelet basic accumulator in case 459 of auxiliary equipment. Auxiliary accumulator 120 is easy replaceable by the use of small door 466 in cover 459 by which is provided against slide out. The auxiliary accumulator 120 removing makes easy spring 125 of accumulator sliding out. Electric linking up contact 245 of auxiliary accumulator 120 provides contacts 307. Board 469 of case 459 auxiliary electronic of charging provides recharging of the cellular phone 451 basic accumulator which is placed in bracelet and with advantage covers electronic circuits of cellular phone 451 in used for bracelet expanding possibility

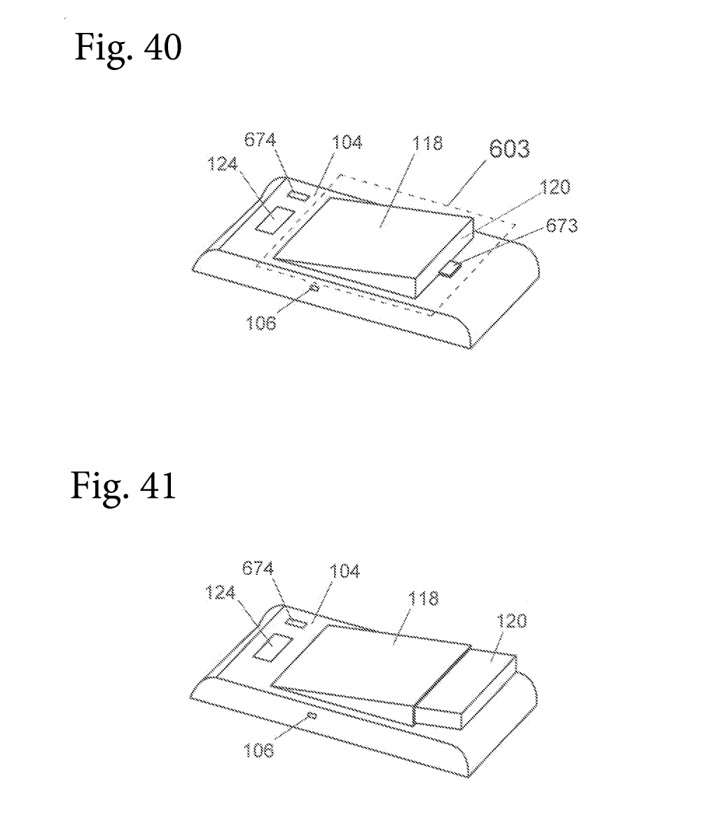

FIG. 40 illustrates partial perspective view of auxiliary equipment 104, slightly opened tipping casing 118 of placement mechanism 603. Auxiliary equipment 104 is fixed instead of back cover of multimedia equipment 600 and is connected by the use of internal connectors. After the sliding closure 673 unlocking the tipping casing 118 of auxiliary accumulator 120 placement mechanism 603, which is part of the auxiliary equipment 104 cover, moderately rises up, the auxiliary accumulator 120 partially slides out, which make possible to take it away and substitute by another charged auxiliary accumulator 120. The auxiliary accumulator 120 is inserted, backwards by the pressing of tipping casing 118. Thanks to this arrangement the exchange of accumulator take some seconds only and is very simple. The placement mechanism 603 covers holes for loudspeaker.

FIG. 41 illustrates partial perspective view of auxiliary equipment 104, slightly opened tipping casing 118 of placement mechanism 603 arid auxiliary accumulator 120 partially slid out. Transparent hole 124 uncovers space for lens of camera, while holes for loudspeaker are with advantage covered by grid.

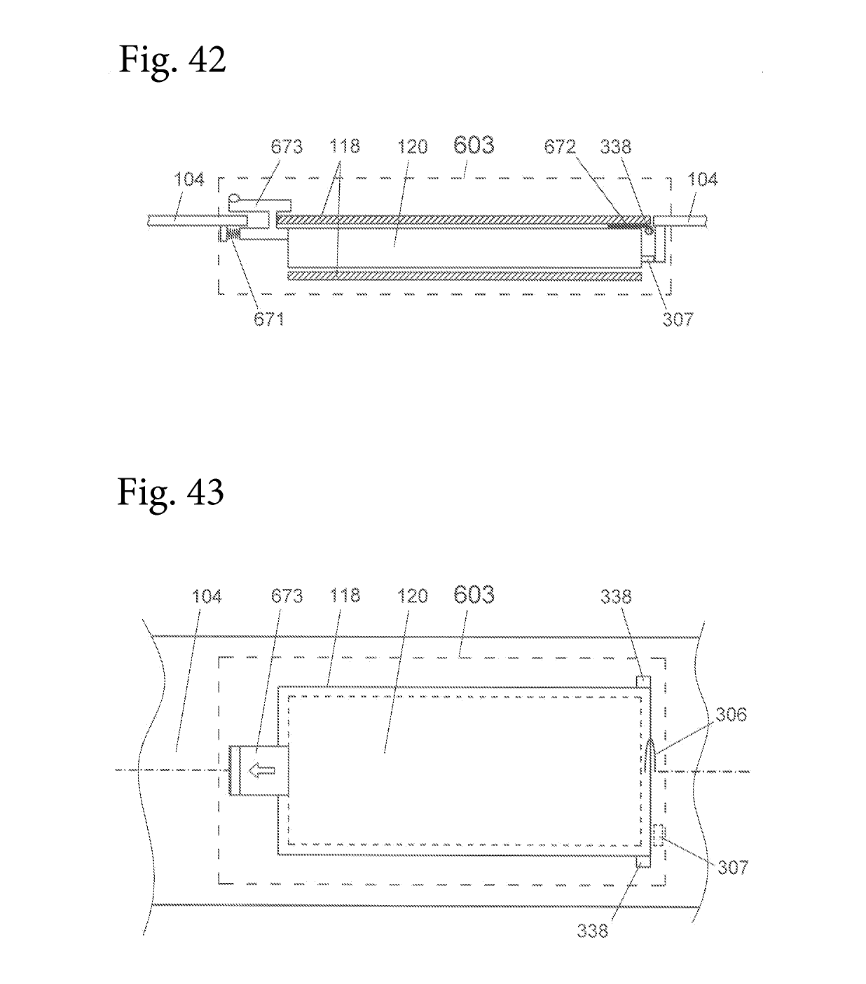

FIG. 42 illustrates operational view to placement of auxiliary accumulator 120. The auxiliary accumulator 120 is placed inside the tipping easing 118, which top side covers the placement mechanism 603. Tipping casing 118 is placed on revolving pin 338 and tipping out by spring 672. The auxiliary accumulator 120 is linked by contacts 307. In the closed position is tipping casing 118 of placement mechanism 603 ensured by sliding closure 673, which is pressed to connector 3027 by the spring 671.

FIG. 43 illustrates the placement mechanism 603 with tipping casing 118 placing in the auxiliary equipment 104. During auxiliary accumulator 120 changing the sliding closure 673 is shifted in the narrow direction against the spring 671. By this is the placement mechanism 603 released, the spring 672 partially rises up the tipping casing 118 of placement mechanism 603 and bronze spring 306 slides out auxiliary accumulator 120. After the auxiliary accumulator 120 changing the sliding closure 673 is shifted backwards against the spring 671, the tipping casing 118 is inserted, back, the sliding closure 673 is released, by which is tipping caring 118 locked and the spring 671 pressed auxiliary accumulator 120 to contacts 307.

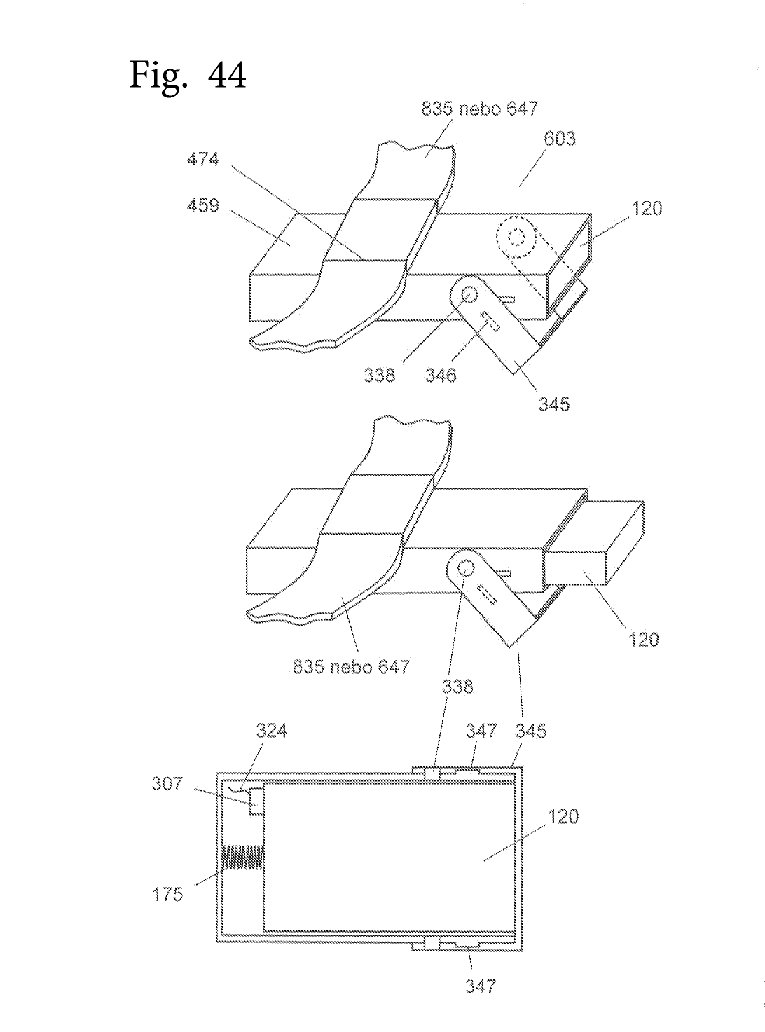

FIG. 44 illustrates another example of the auxiliary accumulator 120 placement mechanism 603 suitable for electronic equipment in bracelet as are chest belt 642 or bracelet 835 mechanical connected by pass through hole 474 for pass through the bracelet or chest belt. This auxiliary accumulator 120 placement mechanism 603 consists of plastic case 459 for auxiliary accumulator 120 inserting. This is resting against flexible contacts 307 for accumulators connection and locked by closure with advantage formed by revolving clamp 345 for the bracelet locking. This is in the closed position locked by latches 346 placed on the arms of revolving clamp, which fit in depressions 347 on the body of plastic case 459. The clamp 345 is turning around revolving pins 338 of plastic case 459. During the auxiliary accumulator 120 changing is revolving cheap 545 lifted up, latches 346 fit out of depressions 347 and unlocked the revolving clamp 345, and the auxiliary accumulator 120 is slid by the spring 175. In the bottom part of placement mechanism is under auxiliary accumulator placed auxiliary board 449 of electronics function extension, which provides all electronics needs. The auxiliary accumulator 120 touchdowns to contacts 307 which are connected by cable 234 to electronic equipment.

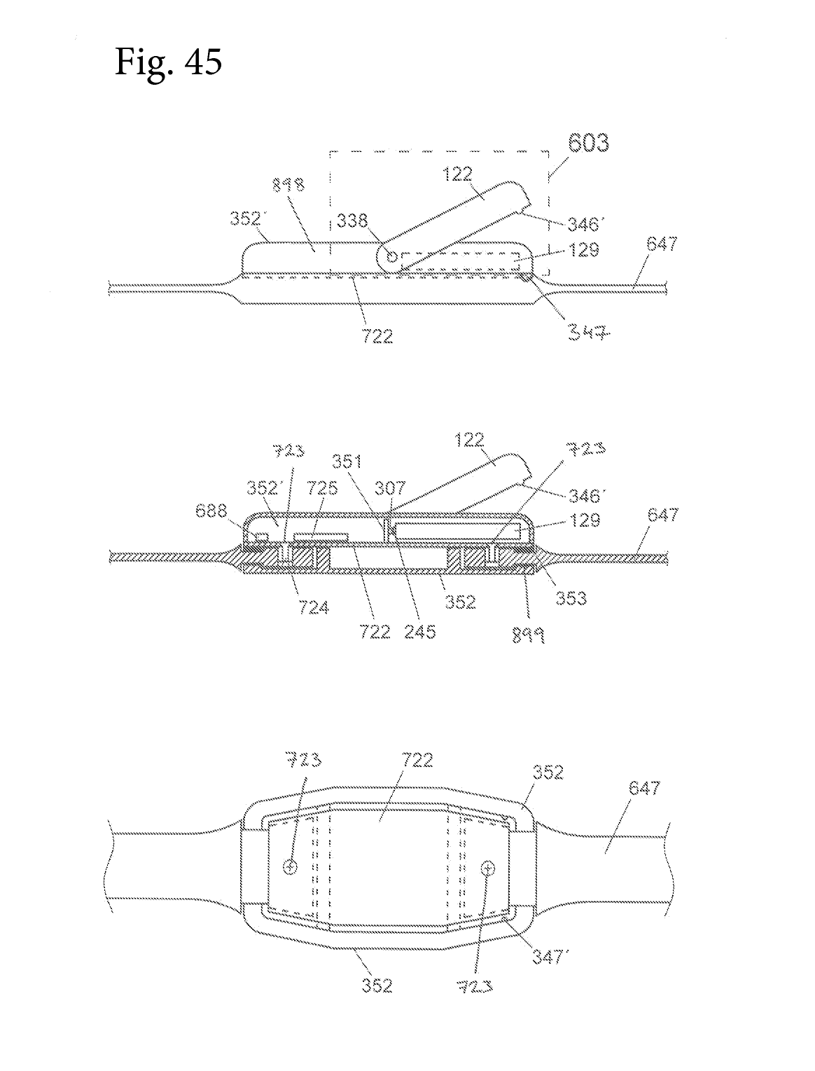

FIG. 45 illustrates another example of the auxiliary accumulator 120 placement mechanism 603 of auxiliary chest belt 647. In the casing 352 of chest belt there is with the exception of active parts of chest belt, with advantage of heart signals monitor 898, placed on the board 722 of chest belt electronic and formed by electronic circuits 725 including bridging accumulator 688 is also placed basic accumulator 129 in placement mechanism 603, which makes possible easy and quickly exchange discharged accumulator and recharged accumulator, with advantage during operation. Basic accumulator 129 is fixed by the press of flexible contacts 327 placed on holders 351 of contacts for accumulator connection to the contact 245 of accumulator 100 by small door 122 on revolving pins 338 closing, which are locked by fitting latches 346′ in depressions 347′. Grooves of both parts of chest belt 647 makes the mechanical connection possible by falling down into outgrowth 899 in the easing 352 of chest belt and on the opposite side into it fits in frets, which are pressed by the hoard 722 of chest belt electronic, which is fixed by screws 723 and nuts 724 of the easing 352.

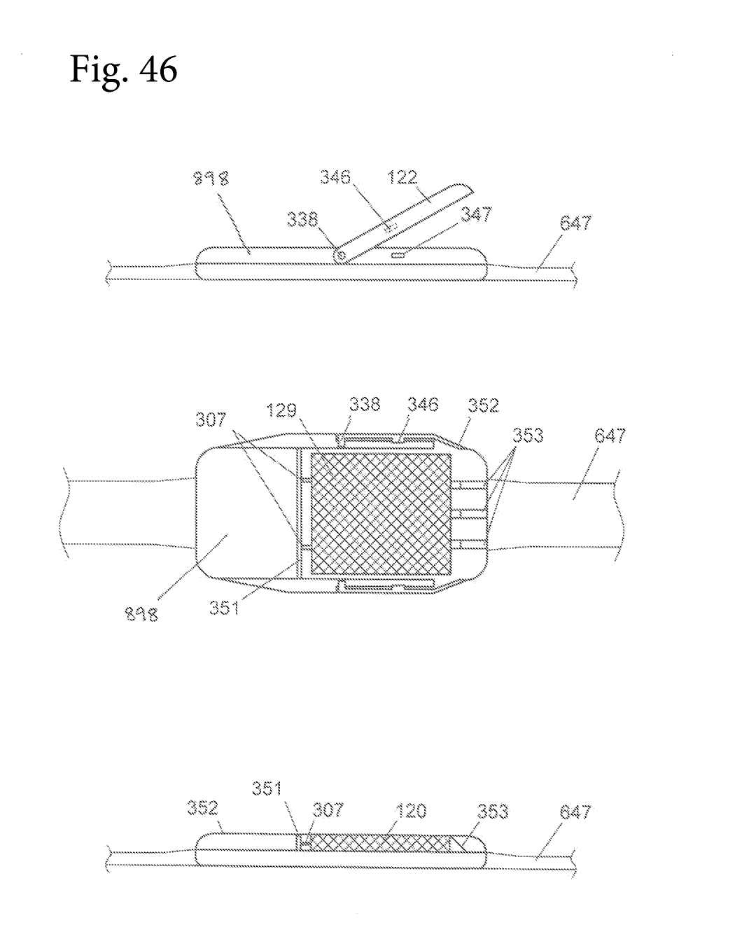

FIG. 46 illustrates configuration of previous variation, the basic accumulator 129 is fixed in different way. It is pressed to contacts 307 by skew frets 353′ dining small door 122 closing.

The small door are locked by two latches Mb placed on inside parts of small door 122 and fit in grooves 347 placed on body of the casing 352. During basic accumulator 129 changing small door uncovers, latches 346 placed on flexible small door slides out of grooves 347, the small door releases and the basic accumulator 129 shifted met by the flexible contacts 307 for the accumulator connection. In the second part of the casing 352 there are placed electronic far voltage modification for active part of electronic including bridging accumulator and communication modules, see FIG. 45 above. The casing 352 of chest belts with advantage includes the bridging source with relevant electronic for the auxiliary accumulator 120 change without operation interruption and monitor of heart signals 898. The advantage in contrast to usual chest belts, which needs to remove the beast signals monitor from chest belt for recharging from the safety reasons, is that recharging and an so the monitor of heart signals removing it is not necessary, because it is done by accumulator changing on the chest belt.

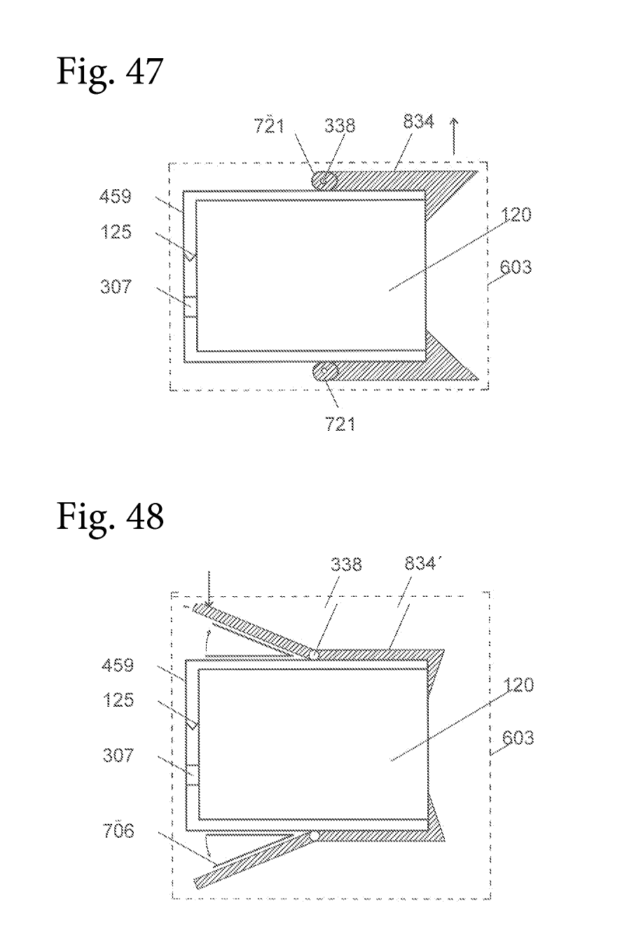

FIG. 47 illustrates placement mechanism 603 with the locking mechanism based on swayed latches 834 revolving around pins 338 and pressed down by coiled spring 721 into starting position. The coiled spring 721 is resting against the case 459 for the accumulator placing and makes possible to be swayed latches 834 deflected in direction of auxiliary equipment to release the auxiliary accumulator 120 by the fingers pushed in the direction of its deflection. After it the coiled spring 721 returned itself into starting position. During insertion of auxiliary accumulator 120 into placement mechanism 603 thanks to the wedge-shaped are swayed latches 834 deflected to the sides, what makes possible to insert the auxiliary accumulator 120 into placement mechanism 603. After the insertion of auxiliary accumulator 120 the compressed coiled spring 721 returns the auxiliary accumulator 120 into starting position and so locked the auxiliary accumulator 120 against sliding by the spring 125 of accumulator sliding out. The auxiliary accumulator 120 can be removed very easy, the fingers of one hand are sufficient can both swayed latches 834 to deflect.

FIG. 48 illustrates next variation of the accumulator placement mechanism by the use of swayed latches 834′ tightly connected to flexible blades 706 function is similar to function of mechanism in the FIG. 47 depicted, the shape of swayed latches 834′ differs only. In the framework of the auxiliary accumulator 120 taking out are both swayed latches 834 by hand pressed and after that flexible blades 706 return v into starting position.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. An auxiliary device to a multimedia equipment,
characterized in that
for extending of the basic features of the multimedia equipment formed by a cellular phone (100), or by a bracelet cellular phone (451), and this of an unmodified multimedia equipment (600), or a modified multimedia equipment (600′), or a compact multimedia equipment (600″), this multimedia equipment is connected with an optionally interchangeable auxiliary device (104), which auxiliary device (104) is adaptable by elements comprised in it for at least one of the following added features, namely for the feature of an easy replacement of an auxiliary accumulator (120) the auxiliary device (104) is optionally provided with a placement mechanism (603) for this easy replacement without any removal of the auxiliary device (104), or without any removal of a cover (101) of the cellular phone (100), or without any removal of another element, or equipment, an further for the feature of a replacement of the auxiliary accumulator (120) without any interruption in operation of the cellular phone (100), and for a full-valued charging of a basic accumulator (129) from the auxiliary accumulator (120), wherein the basic accumulator (129) is fully chargeable also any connecting to the external charger, and this so that the auxiliary device (104) is optionally provided with a module (199) of continuous charging, or with a voltage converter (132). And further for the feature of data processing independently of the electronics of the cellular phone (100), a this so that in case of the unmodified multimedia equipment (600), or the modified multimedia equipment (600) this auxiliary device (104) is provided with an electronics block (608) of the auxiliary device (104) operating independently of the electronics of the cellular phone (100), and in case of the compact multimedia equipment (600″) so that it is provided with a non-disassemblable common electronics board (331), or a replaceable common electronics board (332), or an electronics board (333) of the compact multimedia equipment (600″), and with an independently replaceable electronics board (334) of the auxiliary device (104), wherein the electronics of the auxiliary device (104) and the electronics of the cellular phone (100) are adaptable to operate independently of each other and communicate together, wherein connection of the unmodified multimedia equipment (600) with the auxiliary device (104) is realized by standard fixing elements (602) and connection of the modified multimedia equipment (600′) with the auxiliary device (104) is realized by specifically designed fixing elements (602), and in the compact multimedia equipment (600″) the auxiliary device (104) is integrated, and this as non-disassemblable, or it is placed as disassemblable, or it is adapted for an easy removal.

23. The auxiliary device to a multimedia equipment according to claim 22,
characterized in that
in the unmodified multimedia equipment (600), the fixing elements (602) are formed by a lug (128) of the cover (101), and/or of the auxiliary device (104), with an oppositely positioned depression (127) in the cellular phone (100), or by clips (505), or by flexible lugs (322), and in the modified multimedia equipment (600′) the fixing elements (602) are formed as structurally reinforced ones, and are formed as a lug element (509) comprising a cut-through hollow body (316) provided with a transverse spring (317) for gripping of a dowel (315) provided with a shoulder.

24. The auxiliary device to a multimedia equipment according to claim 22,
characterized in that
it comprises a placement mechanism (603) of the accumulator (120), into which the auxiliary accumulator (120) is optionally inserted, and from which it is extractable, without any taking away of the auxiliary device (104), or of the cover (101) of the multimedia equipment (600, 600′, 600″) through a small door (122), or by using a small handle (128), or by a mechanism (339) for releasing of the accumulator (120), which is reacting to pressure exerted on the auxiliary accumulator (120).

25. The auxiliary device to a multimedia equipment according to claim 22,
characterized in that
for realization of the auxiliary features, supplementing the basic features of the multimedia equipment (600, 600′, 600″), the pieces of auxiliary device (104) are adaptable for various required auxiliary features of the multimedia equipment (600, 600′, 600″) in optional designs and they are optionally attachable to the multimedia equipment (600, 600′, 600″), wherein the multimedia equipment (600, 600′, 600″) without any attached piece of auxiliary device (104) fulfils only the basic features of the cellular phone (100).

26. The auxiliary device to a multimedia equipment according to claim 22,
characterized in that
to realize the auxiliary features supplemental to the basic features of the multimedia equipment (600, 600′, 600″), the pieces of auxiliary device (104) are realized with a stand-alone, at least one microprocessor (133) as supplemental to a basic microprocessor of the multimedia equipment (600, 600′, 600″), and these microprocessors are adaptable for mutually independent operation, and further, they are adaptable for mutual communication.

27. The auxiliary device to a multimedia equipment according to claim 22,
characterized in that
it comprises a module (199) for continuous charging, by means of which the basic accumulator (129) is chargeable with the help of an internal charger (130) of the basic accumulator of the cellular phone (100) from the auxiliary accumulator (120), or the auxiliary accumulator (120) is connected to a voltage converter (132) for converting of the auxiliary accumulator (120) voltage to a voltage value meeting specifications of the charging voltage for the cellular phone (100) connected, and further, by means of contacts of a second USB connector (105) of the auxiliary device (104), which is inserted into a USB connector (102) of the cellular phone (100), it is connected via an internal charger (130) of the basic accumulator (129) of the cellular phone (100) to the basic accumulator (129), whereby it is continuously chargeable, whereby the operation time period of the cellular phone (100) is prolonged, wherein the auxiliary accumulator (120) is removable without any influencing of the cellular phone (100) operation and an external power supply (118) for charging of the auxiliary accumulator (120) and/or the basic accumulator (129) via the charger (131) is connected to the module (199) for continuous charging.

28. The auxiliary device to a multimedia equipment according to claim 22,
characterized in that
by means of fastening elements for fixing of the cover (101) of the cellular phone (100), or of the auxiliary device (104), which are realizable by the fastening elements (509, 602), the auxiliary device (104) to the multimedia equipment (600, 600′) is so mechanically fixed that in handling position of the auxiliary device (104), as well as position of all modules, units, and parts comprised in it, is secured against accidental displacement or turning slightly with regard to the multimedia equipment (600, 600′), wherein for common handling of the cellular phone (100) position of the auxiliary device (104), and of the modules, units and parts comprised in it, with regard to the cellular phone (100) is unchangeable.

29. The auxiliary device to a multimedia equipment according to claim 22,
characterized in that
it is realizable with an auxiliary display (325) of the auxiliary device (104) for displaying of data from the auxiliary device (104), or from the cellular phone (100), wherein the auxiliary display (325) is adaptable for controlling of the auxiliary device (104) and/or of the cellular phone (100), and this for the auxiliary device (104) in connection with the multimedia equipment (600, 600′, 600″) formed by the cellular phone (100) or by the bracelet cellular phone in the unadapted, adapted, or compact designs, or for auxiliary equipment in the bracelet, which is connected to the multimedia equipment (600, 600′, 600″).

30. The auxiliary device to a multimedia equipment according to claim 22,
characterized in that
the cellular phone (100) je provided with the auxiliary device (104) comprising an auxiliary electronics board (123) adapted for processing of ECG signals, heart rate, and/or arrhythmia, wherein the signals are inputted to the auxiliary electronics board (123) from adhered ECG electrode(s) (143) for a one-lead to twelve-lead ECG, which ECG electrode(s) (143) are wire-connectable via a front-end module (141) to a microprocessor (133), or the ECG signals are processed in a chest strap (147) provided with ECG electronics together with the ECG electrode(s) (143) placed on the chest strap (147) and/or externally, or the ECG signals are processed in a chest strap (647) provided with the ECG electrodes (143) by the auxiliary front-end module (141) for a one-lead to twelve-lead ECG, and via a communication module (144), the respective processed signals are transmitted to the auxiliary electronics board (123), more particularly to its transmission module (135), via a wireless connection realizable as a Bluetooth, or an ANT, or a ZigBee module (139) in the basic communication block (149) for a wireless transmission of the heart rate, or arrhythmia, a 5,5 kHz module (134) is applicable for such transmission, wherein, via these modules, the outputs of the auxiliary front-end module (141) are led to an input of the microprocessor (133) to be processed further and displayed on the auxiliary display (325) of the auxiliary equipment, or after transmission of data via the second USB connector (105) the data are displayed on the cellular phone (100) display, wherein the processed outputs from the microprocessor (133) are transmittable further via a module (898) for communication with a cellular data and/or voice network, or via a module (137) for transmission in a WiFi network to a server (806), wherefrom the data is available to authorized users, medical personnel, or a surveillance center.

31. The auxiliary device to a multimedia equipment according to claim 22,
characterized in that
in a mechanical design the auxiliary accumulator (120) with electronics is placed in a case (459) in the upper part (452) of a bracelet, which upper part (452) is further provided with a buckle (455) of a fastening and a belt loop (454) for fastening of a loose end of the bottom part (458) of the bracelet when fastened, where the loose end of the bottom part (458) of the bracelet is provided with a USB plug (457), wherein on the side to the buckle (455) of the fastening the casing (459) is provided with a plug (473) of the USB connector, wherein on the upper part (452) of bracelet the casing (459) is mounted as sliding by means of an opening (474), optionally of the wedge-like shape, in its bottom part for a conductive connection of the plug (457) and the socket (473) of the USB connector, and also a casing (459) is realizable on the upper part (452) of the bracelet, which, owing to an opening (474), optionally of a wedge-like shape, is displaceable into the central part of the bracelet, wherein, alternatively, the auxiliary device (104) is adapted for placing in a bracelet electronic device represented by a bracelet cellular phone (451) and fixing of this auxiliary equipment in this bracelet cellular phone (451) is feasible by means of fastening elements identical with those on the accumulator cover (460) of the cellular phone (451), wherein casing (459) of the auxiliary equipment is both mechanically and electrically connectable with this cellular phone (451) by a replacement with the accumulator cover (460) after its removal when the casing (459) deployable in a position on the upper part (452) of the bracelet, wherein in an alternative fixing of the casing (459) of the auxiliary equipment in the bottom part (458) of the bracelet the auxiliary device (104) is connected with the bracelet electronic device represented by a bracelet cellular phone (451) and with an additional belt loop with the connector (453) placed in the upper part (452) of the bracelet.

32. The auxiliary device to a multimedia equipment according to claim 22,
characterized in that
another casing for placing of a spare auxiliary accumulator (120) and of electronics for charging of the internal basic accumulator of the bracelet cellular phone is placed as removable in the casing (459) of the auxiliary equipment, which another casing is provided with a closable small door (416), wherein a compression spring (125) for sliding off the accumulator acts against the closed small door (466), which door (466) is secured by a snap catch, and the accumulator is connected by contacts (307) with an electronics board for charging of the internal accumulator of the bracelet cellular phone, wherein, the closable small door (466) of the placement mechanism (603) is provided with a door hinge (119) and a door latch (346′), or with a handle (308) with a spring (310) or with a closure (673) with a spring (671), or with a closing clamp (345) with a latch (346), or with a small door (122) with a door latch (346).

33. The auxiliary device to a multimedia equipment according to claim 22,
characterized in that
on the rear side it is provided with a tipping casing (118) for inserting of the auxiliary accumulator (120) into a placing mechanism (603) situated within this auxiliary device (104), wherein the auxiliary device (104) adapted for fastening instead of the rear cover of the multimedia equipment (600, 600′), and it is linked with this cellular multimedia equipment (600, 600′) by connectors inside, and the auxiliary device (104) is adapted to be provided by a transparent opening (124) for optics of the cellular phone (100) a camera in the rear side of the auxiliary device (104), wherein there is also an adaptable opening (674) above a loudspeaker of the cellular phone (100), and a first USB connector (106) for charging or for connecting to a PC and a sliding locking closure (673) of the placement mechanism (603) are realizable in the body of the auxiliary device (104).

34. The auxiliary device to a multimedia equipment according to claim 22,
characterized in that
it is formed by a tipping casing (118) for inserting of the auxiliary accumulator (120), the upper outside side of which forms also a cover of the placement mechanism (603), wherein this tipping casing (118) is mounted on a pivotable pin (338), which pivotable pin (338) is provided by a spring (672) for lifting up, wherein in the closed position the placement mechanism (603) is held by the sliding locking closure (673), which sliding locking closure (673) is pushed by the lock spring (671) against the inserted accumulator (120), and this accumulator (120) is pushed against contacts (307) for connection of the accumulator to the electronics of the cellular multimedia equipment (600), wherein optionally a tipping casing (118) is present, which tipping casing (118) is provided with a bronze spring (306) on its inner back side, next to the contacts (307), for sliding out the auxiliary accumulator (120) during lifting of the tipping casing (118) after the sliding closure (673) has been unlocked.

35. The auxiliary device to a multimedia equipment according to claim 22,
characterized in that
for the bracelet cellular multimedia equipment (600) the casing (459) of the auxiliary equipment is modified as a casing for inserting of the auxiliary accumulator (120), which casing is provided by a coiled spring (175) on its inner rear side, next to the contacts (307), which coiled spring (175) serves for sliding out the auxiliary accumulator (120) during tipping back of the pivotable clamp (345), which clamp (345) is pivotable on pins (338) after unlocking of its latches (346) from the grooves (347) for the latches, wherein on the inner side of the casing (459) wall, under the auxiliary accumulator (120), the electronics board (449) is placed, and on the external side of the casing (459) wall an opening (474) for passing through of the bracelet it is provided.

36. The auxiliary device to a multimedia equipment according to claim 22,
characterized in that
a chest strap (145) is attachable to the auxiliary device (104), wherein the small door (122) of the placement mechanism (603), fastened pivotably to the casing (352) of the chest strap, are adapted to be locked by means of the latches (346) placed on the longitudinal arms of the small door (122) of the placement mechanism (603) ending at the pivotable pin (338) and in the closed position of this small door (122) sunk into the grooves (347) formed in both sides of the casing (352) of the chest strap, wherein on its inner side between both sides this casing (352) of the chest strap is provided with a holder (351) of the contacts (307) of contacting with the auxiliary accumulator (120), and for pressing of the inserted auxiliary accumulator (120) onto the contacts (307) the small door (122) of the placement mechanism (603) is provided with two and more frets (353) for delimiting of the accumulator position, wherein the frets (353) of the small door (122) of the placement mechanism (603) are optionally wedge-shaped or skewed for inserting of the auxiliary accumulator (120) so that in the closed position of the small door (122) of the placemen mechanism (603) the inserted auxiliary accumulator (120) is firmly pressed in by the flexible contacts (307) for pressing of the auxiliary accumulator (122) off the space of its placement when the small door (122) of the placement mechanism (603) is lifted up, wherein in the remaining space of the casing (352) of the chest strap, under the space (348) for placement of the auxiliary accumulator (120), the board (123) of electronics of the auxiliary equipment, including the signaling LED module (134) of transmissions in the 5 kHz band is placed, and on its side walls, the realizable placement mechanism (603) of the accumulator with the casing (459) for placing of the auxiliary accumulator (120) is provided with swayed latches (834, 834) pivotable on a pin (338), when the swayed latches (834) are of wedge-shaped leading edges for an easy sliding of the auxiliary accumulator into the casing (459), wherein gripping of the latches is provided by blades (835), and unlocking of the latches (834′, 834) for sliding out of the auxiliary accumulator (120) is reached by swaying around the pin (338), when sliding of the auxiliary accumulator (120) out of the casing (459) is carried out by means of the flexible contacts (307), wherein the swayed latches (834′) are optionally provided with a lever-like extension on their controlling sides.

37. The auxiliary device to a multimedia equipment according to claim 22,
characterized in that
the auxiliary device (104) is adapted to be placed on the bracelet (458) and is connectable to the electrodes (143) by wires, or on a chest strap (674) wirelessly, and is adapted to be provided with a display (325) for displaying of signals and data from these electrodes and the chest strap, and also with a wireless connection to communicate with the cellular phone (100), so that the signals and the data are displayable also on the cellular phone, and further, the auxiliary device (104) is adapted to be provided with at least one microprocessor (133) adapted for independent data processing by an optional operating system independently of the choice of operating system installed in the mobile phone or in other microprocessors in the auxiliary device (104), which microprocessors are adaptable for optionally provided operating systems and data provided by these microprocessors and by the microprocessor in the cellular phone (100) are mutually transmittable, and further the auxiliary device (104) is adaptable for displaying of data from the chest belt (674) or from the electrodes) (143) not only on the display (325), but also on the server (806), to which server the data is transmitted, wherein the data is displayed continuously, or only the data off limits with a warning, wherein the auxiliary device (104) is equipped optionally with a reset button (111) for resetting of this warning and/or displaying of the data off limits, and/or for data transmitting to the server (806), and further the auxiliary device (104) is equipped optionally with a panic button (110) for transmitting of an emergency call to the server (806), wherein the auxiliary device (104) is equipped optionally with a memory card for saving of the processed data at least from the electrodes (143) or the strip (147, 674), optionally to the ECG, so that the auxiliary equipment optionally fulfills the functions as a Holter ECG, optionally simultaneously with displaying on the auxiliary device (104) and/or the multimedia equipment (600, 600′, 600″), and/or with their transmission to the server (806), so that the auxiliary device (104) is adaptable for combined operation as a Holter ECG, local displaying for the person monitored, and for remote monitoring of persons by the central control on a PC connectable to the server (806) or by other selected participants via the Internet, wherein the auxiliary device (104) with the chest belt (147, 674) is further provided optionally for simultaneous transmission of data from this chest strap, optionally via a wireless inductive link 5.3 kHz, and simultaneously also via a high-frequency circuit, optionally a Bluetooth, a Bluetooth Low Energy, a Zig Bee, an ANT, a WiFi on a device receiving this signal via one of these wireless connections, optionally to a wrist watch for athletes, placed on a wrist, which wrist watch is provided optionally for displaying of arrhythmia, and/or on the auxiliary device (104), and/or on the cellular phone (100), and data transmitted from the auxiliary device (104) or the multimedia equipment (600, 600′, 600″) via the wireless link to the server (806) are displayed optionally on the PC of the central control, and/or remotely via the Internet (PH) to the selected participants.

38. The auxiliary device to a multimedia equipment according to claim 22,
characterized in that
its placement mechanism (603), or the mechanism (253) of removing the accumulator is provided with lift-off or sliding small door, or with lift-off or sliding cover as a whole, or of its part only, or the whole auxiliary device (104) is adapted for lift-off or sliding during placement or replacement of the basic (129) or the auxiliary (120) accumulator.

39. The auxiliary device to a multimedia equipment according to claim 22,
characterized in that
the auxiliary equipment (104″a, 104″b) is integrated in the multimedia equipment (600″) and it is placed in it as non-removable, removable, or replaceable by means of a mechanism (323) for placing of the block for easy removal, and in case of the removable placing, the board (334) of electronics of the auxiliary equipment is replaceable independently in the space (330) for inserting, or in case of the replaceable common board (332) of electronics, when the auxiliary equipment is placed on this board (332) together with the board of electronics of the multimedia equipment, this board is replaceable as a whole, and it is interconnected with the multimedia equipment (600″) by means of a connector (335), or in case of a stand-alone replaceable board (334) of electronics this board (334) is replaceable individually, and is interconnected with the multimedia equipment (600″) by means of the connecting connector (335).

40. The auxiliary device to a multimedia equipment according to claim 22,
characterized in that
the mechanism for connecting of the auxiliary device (104) with the cover (101) of the multimedia equipment (600) represented by the cellular phone (100) is formed by a spacing elastic pad (628) for compensating of unevenness or of bulging of the cover (101), of the bottom plan shape of the auxiliary device (104), which elastic pad (628) is placed between the auxiliary device (104) and the cover (101), wherein mechanical connection of the auxiliary device (104) with the cover (101) is carried out by a screw (631) or by sticking to the cover of the multimedia equipment, and their electric interconnecting is carried out by an interconnecting element represented by an interconnecting cable (319) for interconnecting of the board (123) of the auxiliary equipment electronics with the multimedia equipment (600).

41. The auxiliary device to a multimedia equipment according to claim 22,
characterized in that
the compact multimedia equipment (600′), optionally embodied by the cellular phone (100), comprises the non-dismountable auxiliary equipment (104″b), which is connected with modules of the compact multimedia equipment (600″) placed on the non-dismountable electronics board (331) or on the board (332), which is replaceable as a unit, or the auxiliary equipment (104″b) is placed as allowing disassembly in the compact multimedia equipment (600″) on an independently replaceable electronics board (334) of the auxiliary equipment, optionally via an attaching connector (335), or the unmodified multimedia equipment (600) is connected to the auxiliary device (104) via the connecting elements (601), wherein all electronics of the enlarging features and/or the auxiliary accumulator are comprised in this auxiliary device (104), or for the modified multimedia equipment (600′) the auxiliary device (104) is connected by the connecting elements (601), using modifications of the multimedia equipment (600) with various modules, units, and parts for broadening by adding additional features, which features are optionally partially comprised in the multimedia equipment.
